(19) United States
(12) Patent Application Publication
(10) Pub. No.: US 2024/0319084 A1
(43) Pub. Date: Sep. 26, 2024

(54) RAPID DETERMINATION OF DISEASE IN SURROGATE CELLS USING INFRARED LIGHT

(71) Applicant: The Regents of the University of California, Oakland, CA (US)

(72) Inventors: Cynthia T. McMurray, Oakland, CA (US); James B. Brown, Oakland, CA (US); Edward S. Barnard, Oakland, CA (US); Lila Lovergne, Oakland, CA (US); Dhruba Ghosh, Oakland, CA (US); Aris A. Polyzos, Oakland, CA (US); Renaud Schuck, Oakland, CA (US)

(21) Appl. No.: 18/579,809

(22) PCT Filed: Jul. 15, 2022
(86) PCT No.: PCT/US2022/037364

§ 371 Date: Jan. 16, 2024

Related U.S. Application Data
(60) Provisional application No. 63/222,940, filed on Jul. 16, 2021.

(51) Int. Cl. G01N 21/35; G01N 33/50

(52) U.S. Cl. CPC G01N 21/35; G01N 33/5091; G01N 2021/3595

(57) ABSTRACT

Disclosed herein include systems, devices, and methods for determining a state of a subject (e.g., whether the subject has a disease or is responsive to a treatment of a disease) using FTIR spectral phenotyping before the subject has any symptoms or overt symptoms.

### CROSS-REFERENCE TO RELATED APPLICATIONS

The present application is a U.S. national phase application under 35 U.S.C. § 371 of International Application No. PCT/US2022/037364, filed on Jul. 15, 2022 and published as WO 2023/288096 A1 on Jan. 19, 2023, which claims the benefit of priority to U.S. Patent Application No. 63/222,940, filed Jul. 16, 2021, the content of which is incorporated herein by reference in its entirety.

### STATEMENT REGARDING FEDERALLY SPONSORED R&D

This invention was made with government support under grant numbers R01NS060115, R01GM119161, and R21AG070972, awarded by National Institute of Health; and DE-AC02-05CH11231 awarded by U.S. Department of Energy. The government has certain rights in the invention.

### BACKGROUND

### Field

This disclosure relates generally to the field of phenotyping, and more particularly to spectral phenotyping.

### Background

Some neurodegenerative diseases can be identified by behavioral characteristics relatively late in disease progression. There is currently no method or biomarker to predict who has developed or will develop a disease before the onset of symptoms, when the onset will occur, or the outcome of therapeutics. New methods and biomarkers are needed.

### SUMMARY

Disclosed herein include methods for determining a state of a test subject. In some embodiments, a method for determining a state of a test subject can be under control of a processor (e.g., a hardware processor or a virtual processor). The method can comprise: generating a plurality of reference Fourier transform infrared spectroscopy (FTIR) spectra (e.g., absorption spectra) for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples and the average test FTIR spectrum into a first cluster and a second cluster corresponding to the first state and the second state, respectively. The method can comprise: determining the test sample is in the first state or the second state based on whether the average test FTIR spectrum is in the first cluster or the second cluster.

Disclosed herein include methods for determining a state of a test subject. In some embodiments, a method for determining a state of a test subject is under control of a processor (e.g., a hardware processor or a virtual processor). The method can comprise: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples into a first cluster and a second cluster corresponding to the first state and the second state, respectively (e.g., in a reduced dimensionality space). The method can comprise: determining the test sample is in the first state or the second state based on a first distance between the average test FTIR spectrum and the first cluster and a second distance between the average test FTIR spectrum and the second cluster (e.g., in the reduced dimensionality space). The method can comprise: determining the test sample is in the first state or the second state based on the states of k-nearest neighbors of the average test FTIR spectrum (e.g., in the reduced dimensionality space).

In some embodiments, each of the plurality of reference samples and the test sample comprises about 100 cells to about 1000 cells. Each of the plurality of reference samples and the test sample can comprise about the same number of cells. In some embodiments, the sample comprises a tissue sample. The tissue sample can be about 10 m in thickness. The tissue sample can comprise one layer of cells. In some embodiments, the sample comprises surrogate cells. The surrogate cells can comprise accessible cell types, epithelial cells, fibroblasts, lymphoblasts, peripheral cells, non-neural cells, buccal cells, induced pluripotent stem cells, or a combination thereof.

In some embodiments, the plurality of reference samples and the test sample comprise fixed cells on slides. In some embodiments, the plurality of reference samples and the test sample were prepared in an identical manner. Preparation conditions of the plurality of reference samples and preparation conditions of the test sample were matched (e.g., in terms of the storage temperature, slide preparation and coating). In some embodiments, the slides comprise Calcium fluoride (CaF₂) or silicon (Si) slides. The slides can comprise no coating. The slides can comprise a coating. The coating can comprise poly-L-ornithine (PLO). The coating can comprise wet PLO or dry PLO. In some embodiments, the slides were previously stored at room temperature or −80° C. for up to two weeks prior to capturing of spectra. In some embodiments, the plurality of first reference samples comprises at least 10 samples. The plurality of second reference samples can comprise at least 10 samples.

In some embodiments, the plurality of reference FTIR spectra for each of the plurality of samples and the plurality of test FTIR spectra were captured in an identical manner. Capturing conditions of the plurality of reference FTIR spectra for each of the plurality of samples and capturing conditions the plurality of test FTIR spectra were matched (e.g., in terms of capturing temperature, capturing duration, capturing instrument). In some embodiments, generating the plurality of reference FTIR spectra for each of the plurality of samples and the plurality of test FTIR spectra comprises capturing the plurality of reference FTIR spectra for each of the plurality of samples and the plurality of test FTIR spectra at room temperature or −80° C.

In some embodiments, the first state comprises a first phenotype (e.g., non-diseased or non-responsive), and the second state comprises a second phenotype (e.g., diseased or responsiveness). The first state can be non-responsiveness to a treatment of a disease, and the second state can be responsiveness to the treatment of the disease. The first state can be a non-diseased state, and the second state can be a diseased state. The disease can be a disease subtype. The disease can be a neurological disease, a neurodegenerative disease, a late onset disease, or a cancer. The neurological disease or the neurodegenerative disease can comprise Alzheimer's disease, Huntington's disease, or Fragile X syndrome.

In some embodiments, the one or more characteristics of the test subject and the reference subjects that are matched comprise age, gender, lifestyle, diet, health, ethnicity, and/or medical background (e.g., cholesterol level). In some embodiments, the second reference subjects have no symptoms, have no overt symptoms, is pre-symptomatic, and/or is pre-disease onset.

In some embodiments, the plurality of reference FTIR spectra, the average reference FTIR spectra, the plurality of test FTIR spectra, and the average test FTIR spectra comprise second derivative absorbance spectra. In some embodiments, the plurality of reference FTIR spectra, the average reference FTIR spectra, the plurality of test FTIR spectra, and the average test FTIR spectra comprise spectra between 3050-2800 cm⁻¹ and/or 1800-900 cm⁻¹. In some embodiments, the plurality of reference FTIR spectra for each of the plurality of reference samples and the plurality of test FTIR spectra comprise FTIR spectra generated from whole cells. In some embodiments, the plurality of reference FTIR spectra for each of the plurality of reference samples and the plurality of test FTIR spectra comprise FTIR spectra generated from cytoplasm of cells. In some embodiments, the method comprises segmenting the plurality of reference FTIR spectra for each of the plurality of reference samples and the plurality of test FTIR spectra to determine reference FTIR spectra of the plurality of reference FTIR spectra for each of the plurality of reference samples and test FTIR spectra of the plurality FTIR spectra generated from cytoplasm of cells. The segmenting can be based on integrated absorbance frequencies between 1670-1630 cm⁻¹.

In some embodiments, the method comprises quality testing the plurality of reference FTIR spectra for each of the plurality of samples and the plurality of test FTIR spectra to generate a plurality of quality-tested, reference FTIR spectra for each of the plurality of samples and the plurality of quality-tested, test FTIR spectra. Determining the average reference FTIR spectrum of the plurality of reference FTIR spectra for each of the plurality of reference samples can comprise determining an average reference FTIR spectrum of the plurality of quality-tested, reference FTIR spectra for each of the plurality of reference samples. Determining the average test FTIR spectrum can comprise determining the average test FTIR spectrum of the plurality of quality-tested, test FTIR spectra.

In some embodiments, the method comprises pre-processing the plurality of reference FTIR spectra for each of the plurality of samples and the plurality of test FTIR spectra to generate a plurality of pre-processed, reference FTIR spectra for each of the plurality of samples and the plurality of pre-processed, test FTIR spectra. Determining the average reference FTIR spectrum of the plurality of reference FTIR spectra for each of the plurality of reference samples can comprise determining an average reference FTIR spectrum of the plurality of pre-processed, reference FTIR spectra for each of the plurality of reference samples. Determining the average test FTIR spectrum can comprise determining the average test FTIR spectrum of the plurality of pre-processed, test FTIR spectra. Pre-processing can comprise smoothing, baseline correction, spectral contrast optimization, and/or vector normalization.

In some embodiments, the plurality of reference FTIR spectra for each of the plurality of reference samples and the plurality of test FTIR spectra comprise normalized second derivative spectra. In some embodiments, clustering the average reference FTIR spectra of the plurality of reference samples comprises dimensionality reduction. Clustering the average reference FTIR spectra of the plurality of reference samples can comprise unsupervised clustering. The unsupervised clustering comprises Principal Component Analysis (PCA) and Uniform Manifold Approximation and Projection (UMAP) analysis.

In some embodiments, a Silhouette score of the test sample being determined to be in the first state or the second state is about 0.4 to 0.9. Sensitivity of the test sample being determined to be in the first state or the second state can be at least 0.8. Specificity of the test sample being determined to be in the first state or the second state can be at least 0.8. Accuracy of the test sample being determined to be in the first state or the second state can be at least 0.8.

In some embodiments, the average test FTIR spectrum is in the first cluster if a first distance between the average test FTIR spectrum and the first cluster is shorter than a second distance between the average test FTIR spectrum and the second cluster. The average test FTIR spectrum is in the first cluster if a first distance between the average test FTIR spectrum and the first cluster is longer than a second distance between the average test FTIR spectrum and the second cluster.

In some embodiments, the first distance between the average test FTIR spectrum and the first cluster comprises the first distance between the average test FTIR spectrum and a center of the first cluster. The second distance between the average test FTIR spectrum and the second cluster can comprise the second distance between the average test FTIR spectrum and a center of the second cluster. In some embodiments, the first distance between the average test FTIR spectrum and the first cluster comprises the first distance between the average test FTIR spectrum and k-nearest neighbors of the first cluster. The second distance between the average test FTIR spectrum and the second cluster comprises the second distance between the average test FTIR spectrum and k-nearest neighbor of the second cluster. k can be 10.

Disclosed herein include systems for determining a state of a test subject. In some embodiments, a system for determining a state of a test subject comprises: non-transitory memory configured to store executable instructions. The system can comprise: a processor (e.g., a hardware processor or a virtual processor) in communication with the non-transitory memory, the hardware processor programmed by the executable instructions to perform: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The processor can be programmed by the executable instructions to perform: determining an average reference FTIR spectrum of the plurality of reference FTIR spectra for each of the plurality of reference samples. The processor can be programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples and the average test FTIR spectrum into a first cluster and a second cluster corresponding to the first state and the second state, respectively. The processor can be programmed by the executable instructions to perform: determining the test sample is in the first state or the second state based on whether the average test FTIR spectrum is in the first cluster or the second cluster.

Disclosed herein include systems for determining a state of a test subject. In some embodiments, a system for determining a state of a test subject comprises: non-transitory memory configured to store executable instructions and an average reference Fourier transform infrared spectroscopy (FTIR) spectrum of a plurality of reference FTIR spectra for each of a plurality of reference samples. The plurality of reference samples can comprise a plurality of first reference samples obtained from first reference subjects known to be in a first state and a plurality of second reference samples obtained from reference subjects known to be a second state. The system can comprise: a hardware processor in communication with the non-transitory memory, the hardware processor programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples and the average test FTIR spectrum into a first cluster and a second cluster corresponding to the first state and the second state, respectively. The processor can be programmed by the executable instructions to perform: determining the test sample is in the first state or the second state based on whether the average test FTIR spectrum is in the first cluster or the second cluster.

Disclosed herein include systems for determining a state of a test subject. In some embodiments, a system for determining a state of a test sample comprises: non-transitory memory configured to store executable instructions. The system can comprise: a hardware processor in communication with the non-transitory memory, the hardware processor programmed by the executable instructions to perform: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The processor can be programmed by the executable instructions to perform: determining an average reference FTIR spectrum of the plurality of reference FTIR spectra for each of the plurality of reference samples. The processor can be programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples into a first cluster and a second cluster corresponding to the first state and the second state, respectively, in a reduced dimensionality space. The processor can be programmed by the executable instructions to perform: determining the test subject is in the first state or the second state based on a first distance between the average test FTIR spectrum and the first cluster and a second distance between the average test FTIR spectrum and the second cluster.

Disclosed herein include systems for determining a state of a test sample. In some embodiments, a system for determining a state of a test sample comprises: non-transitory memory configured to store executable instructions and an average reference Fourier transform infrared spectroscopy (FTIR) spectrum of a plurality of reference FTIR spectra for each of a plurality of reference samples. The plurality of reference samples can comprise a plurality of first reference samples obtained from first reference subjects known to be in a first state and a plurality of second reference samples obtained from reference subjects known to be a second state. The system can comprise: a hardware processor in communication with the non-transitory memory, the hardware processor programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples into a first cluster and a second cluster corresponding to the first state and the second state, respectively, in a reduced dimensionality space. The processor can be programmed by the executable instructions to perform: determining the test sample is in the first state or the second state based on a first distance between the average test FTIR spectrum and the first cluster and a second distance between the average test FTIR spectrum and the second cluster.

In some embodiments, each of the plurality of reference samples and the test sample comprises about 100 cells to about 1000 cells. Each of the plurality of reference samples and the test sample can comprise about the same number of cells. In some embodiments, the sample comprises a tissue sample. The tissue sample can be about 10 m in thickness. The tissue sample can comprise one layer of cells. In some embodiments, the sample comprises surrogate cells. The surrogate cells can comprise accessible cell types, epithelial cells, fibroblasts, lymphoblasts, peripheral cells, non-neural cells, buccal cells, induced pluripotent stem cells, or a combination thereof.

In some embodiments, the plurality of reference samples and the test sample comprise fixed cells on slides. In some embodiments, the plurality of reference samples and the test sample were prepared in an identical manner. Preparation conditions of the plurality of reference samples and preparation conditions of the test sample were matched (e.g., in terms of the storage temperature, slide preparation and coating). In some embodiments, the slides comprise Calcium fluoride (CaF₂) or silicon (Si) slides. The slides can comprise no coating. The slides can comprise a coating. The coating can comprise poly-L-ornithine (PLO). The coating can comprise wet PLO or dry PLO. In some embodiments, the slides were previously stored at room temperature or −80° C. for up to two weeks prior to capturing of spectra. In some embodiments, the plurality of first reference samples comprises at least 10 samples. The plurality of second reference samples can comprise at least 10 samples.

In some embodiments, the plurality of reference FTIR spectra for each of the plurality of samples and the plurality of test FTIR spectra were captured in an identical manner. Capturing conditions of the plurality of reference FTIR spectra for each of the plurality of samples and capturing conditions the plurality of test FTIR spectra were matched (e.g., in terms of capturing temperature, capturing duration, capturing instrument, or IR intensity). In some embodiments, generating the plurality of reference FTIR spectra for each of the plurality of samples and the plurality of test FTIR spectra comprises capturing the plurality of reference FTIR spectra for each of the plurality of samples and the plurality of test FTIR spectra at room temperature or −80° C.

In some embodiments, the first state comprises a first phenotype (e.g., non-diseased or non-responsive), and the second state comprises a second phenotype (e.g., diseased or responsiveness). The first state can be non-responsiveness to a treatment of a disease, and the second state can be responsiveness to the treatment of the disease. The first state can be a non-diseased state, and the second state can be a diseased state. The disease can be a disease subtype. The disease can be a neurological disease, a neurodegenerative disease, a late onset disease, or a cancer. The neurological disease or the neurodegenerative disease can comprise Alzheimer's disease, Huntington's disease, or Fragile X syndrome.

In some embodiments, the one or more characteristics of the test subject and the reference subjects that are matched comprise age, gender, life style, diet, health, ethnicity, and/or medical background (e.g., cholesterol level). In some embodiments, the second reference subjects have no symptoms, have no overt symptoms, is pre-symptomatic, and/or is pre-disease onset.

In some embodiments, the plurality of reference FTIR spectra, the average reference FTIR spectra, the plurality of test FTIR spectra, and the average test FTIR spectra comprise second derivative absorbance spectra. In some embodiments, the plurality of reference FTIR spectra, the average reference FTIR spectra, the plurality of test FTIR spectra, and the average test FTIR spectra comprise spectra between 3050-2800 cm⁻¹ and/or 1800-900 cm⁻¹. In some embodiments, the plurality of reference FTIR spectra for each of the plurality of reference samples and the plurality of test FTIR spectra comprise FTIR spectra generated from whole cells. In some embodiments, the plurality of reference FTIR spectra for each of the plurality of reference samples and the plurality of test FTIR spectra comprise FTIR spectra generated from cytoplasm of cells. In some embodiments, the processor is programmed by the executable instructions to perform: segmenting the plurality of reference FTIR spectra for each of the plurality of reference samples and the plurality of test FTIR spectra to determine reference FTIR spectra of the plurality of reference FTIR spectra for each of the plurality of reference samples and test FTIR spectra of the plurality FTIR spectra generated from cytoplasm of cells. The segmenting can be based on integrated absorbance frequencies between 1670-1630 cm⁻¹.

In some embodiments, the processor is programmed by the executable instructions to perform: quality testing the plurality of reference FTIR spectra for each of the plurality of samples and the plurality of test FTIR spectra to generate a plurality of quality-tested, reference FTIR spectra for each of the plurality of samples and the plurality of quality-tested, test FTIR spectra. Determining the average reference FTIR spectrum of the plurality of reference FTIR spectra for each of the plurality of reference samples can comprise determining an average reference FTIR spectrum of the plurality of quality-tested, reference FTIR spectra for each of the plurality of reference samples. Determining the average test FTIR spectrum can comprise determining the average test FTIR spectrum of the plurality of quality-tested, test FTIR spectra.

In some embodiments, the processor is programmed by the executable instructions to perform: pre-processing the plurality of reference FTIR spectra for each of the plurality of samples and the plurality of test FTIR spectra to generate a plurality of pre-processed, reference FTIR spectra for each of the plurality of samples and the plurality of pre-processed, test FTIR spectra. Determining the average reference FTIR spectrum of the plurality of reference FTIR spectra for each of the plurality of reference samples can comprise determining an average reference FTIR spectrum of the plurality of pre-processed, reference FTIR spectra for each of the plurality of reference samples. Determining the average test FTIR spectrum can comprise determining the average test FTIR spectrum of the plurality of pre-processed, test FTIR spectra. Pre-processing can comprise smoothing, baseline correction, spectral contrast optimization, and/or vector normalization.

In some embodiments, the plurality of reference FTIR spectra for each of the plurality of reference samples and the plurality of test FTIR spectra comprise normalized second derivative spectra. In some embodiments, clustering the average reference FTIR spectra of the plurality of reference samples comprises dimensionality reduction. Clustering the average reference FTIR spectra of the plurality of reference samples can comprise unsupervised clustering. The unsupervised clustering comprises Principal Component Analysis (PCA) and Uniform Manifold Approximation and Projection (UMAP) analysis.

In some embodiments, a Silhouette score of the test sample being determined to be in the first state or the second state is about 0.4 to 0.9. Sensitivity of the test sample being determined to be in the first state or the second state can be at least 0.8. Specificity of the test sample being determined to be in the first state or the second state can be at least 0.8. Accuracy of the test sample being determined to be in the first state or the second state can be at least 0.8.

In some embodiments, the average test FTIR spectrum is in the first cluster if a first distance between the average test FTIR spectrum and the first cluster is shorter than a second distance between the average test FTIR spectrum and the second cluster. The average test FTIR spectrum is in the first cluster if a first distance between the average test FTIR spectrum and the first cluster is longer than a second distance between the average test FTIR spectrum and the second cluster.

In some embodiments, the first distance between the average test FTIR spectrum and the first cluster comprises the first distance between the average test FTIR spectrum and a center of the first cluster. The second distance between the average test FTIR spectrum and the second cluster can comprise the second distance between the average test FTIR spectrum and a center of the second cluster. In some embodiments, the first distance between the average test FTIR spectrum and the first cluster comprises the first distance between the average test FTIR spectrum and k-nearest neighbors of the first cluster. The second distance between the average test FTIR spectrum and the second cluster comprises the second distance between the average test FTIR spectrum and k-nearest neighbor of the second cluster. k can be 10.

Disclosed herein include embodiments of a computer readable medium. In some embodiments, a computer readable medium comprising executable instructions, when executed by a processor (e.g., a hardware processor or a virtual processor) of a computing system or a device, cause the processor, to perform any method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIGS. 1A-1B. Concept of cell phenotyping by infrared spectroscopy. FIG. 1A. Schematic of a representative infrared spectrum of astrocytes and the attribution of the prominent chemical features between 4000-900 cm⁻¹. AA/I/II: amide A/I/II, v: stretching, δ: bending, as: asymmetric, s: symmetric vibrations. FIG. 1B. Brief outline of the analysis pipeline for spectral phenotyping, as discussed in example 1. After 7-10 days, cells were plated and cultured overnight onto IR compatible calcium fluoride (CaF₂) substrates, fixed and dried before the spectral analysis. A representative brightfield and corresponding IR image of astrocytes are displayed. IR images were reconstructed on the amide I band (AI) for optimal background/cell contrast. Each tile comprises 128 by 128 pixels (5.5 μm²), each of which contains a FTIR spectrum (in blue), thus constituting hyperspectral images. The raw spectral images were carried through three processing steps to generate a cell signature. (Segmentation) The cells were segmented to extract from IR images the nucleus, cytoplasm, and whole cell raw spectra. (Pre-processing) Raw spectra were pre-processed to generate normalized second derivative spectra (Classification and statistics). Statistical analysis was used to evaluate the disease classification using Principal Component Analysis (PCA) and Uniform Manifold Approximation and Projection (UMAP) analysis. Scale bar=100 μm.

FIGS. 2A-2E. HD mothers and their pups display no overt pathology relative to WT animals. FIG. 2A. Schematic summary of behavior in HdhQ(150/150) animals with age. The P2 pups, their mothers (12 weeks), and symptomatic 2-year animals are displayed on the timeline. FIG. 2B. Cartoon depicting an adult striatum in red and the white box indicating the regions probed in the brain slices in FIG. 2C. FIG. 2C. Mouse striatal brain sections were analyzed for neurons (NeuN antibody) alone, astrocytes (GFAP antibody) alone or as a merged image (Merge) of the two. The striatal regions were compared between WT and HD animals at various ages. There were no differences in neuronal counts in the striatum of HD animals compared to WT, except at very late ages (2 years of age). There was no significant difference in astrocyte levels (GFAP intensity per field) between HD and WT at any age. Scale bar is 50 μm. FIGS. 2D-2E. Quantification of neuronal counts and astrocyte counts from FIG. 2C. ** p-value:<0.005 (Student's t-test, 2 tailed, equal variance homoscedastic).

FIG. 3A. Grip test for motor function. The time in seconds is a measure of duration for gripping the bar (right). Performance is plotted as time (sec) versus age (wks) in WT and HD animals (left). The WT and HD animals had similar grip performance up to 60 weeks. n=16; * p-value: <0.05; ** p-value: <0.005 (Student's t-test, 2 tailed, equal variance homoscedastic).

FIG. 3B. (left) Fluorescence staining of astrocytes with Mitotracker Green (green) to visualize mitochondria number and activity, which were equivalent in WT and HD cells. DAPI staining (blue) indicates the position of the nucleus. To the right is quantification of mitochondrial staining in astrocyte cultures from the CBL or the STR, as indicated. Light gray is WT and dark gray is HD; n=50 (right). Variance is reported as standard error. The scale bar is 10 μm.

FIG. 3C. Full length uncropped western gels of normal and mutant huntingtin protein corresponding to the cropped images in FIG. 4F. (Left) Total protein loading control for the WT and HD animals in the cerebellum (CBL) and striatum (STR), as indicated, visualized with No-Stain Protein Labelling Reagent (Thermofisher). The boxed region corresponds to the four lanes in the gels on the right. (Right) The nitrocellulose blots were probed with an anti-Htt antibody (upper blot), to the normal huntingtin protein in the WT or to the faster migrating band in the heterozygous HD sample. The anti-polyQ antibody (lower blot) primarily detects the mutant protein in the slower migrating band in the HD sample.

FIGS. 4A-4F. Astrocyte cultures from WT and HD animals are visually indistinguishable. FIG. 4A. Astrocyte cell lines from CBL, STR, CTX were dissociated and isolated from the brains of postnatal (P2) mice, from either WT or HD mice. FIG. 4B. Cartoon showing the developing mouse brain at P4 and the dissected regions used in the analysis. The regions are schematically illustrated is the Nissl-stained brain image (purple) from P4 animals. FIG. 4C. A representative brightfield image of primary astrocytes from the cortex of WT mice. FIG. 4D. Purified SV40T astrocytes in all 3 brain regions from WT and HD mice. Scale bars=20 μm. FIG. 4E. Transformed cultures were stained for Glutamate Aspartate Transporter 1 (GLAST1) antibody marker to confirm their identity as astrocytes, as well as stained with DAPI to define the nucleus. Scale bars=20 μm. Cell lines of either genotype had similar morphology. FIG. 4F. Western blot analysis showing that mouse astrocytes from WT and HD mice express normal htt and the mutant (mhtt), respectively, in the STR and CBL. HD astrocytes alone express mhtt, which includes an expanded polyQ stretch. The loading control is total protein visualized with No-Stain Protein Labelling Reagent. The uncropped images are shown in FIG. 3C.

FIGS. 5A-5K. Segmentation reveals differences in the lipid features in the WT and HD astrocytes FTIR signatures. Local Ostu's filter was applied to determine the background from the entire cell (FIG. 5A) or nucleus (FIG. 5B, shown in magenta). Seed points were used to localize cells from their estimated center (FIG. 5C, red dots). Seed watershed segmentation was applied to whole cells (FIG. 5D) and nuclei (FIG. 5E). Seed watershed segmentation was applied to the cytoplasm of the cells (FIG. 5F, entire cell pixels minus nucleus pixels). Scale bars=100 μm. An example of raw extracted whole astrocyte mean spectra before (left of FIG. 5G) and after (right of FIG. 5G) quality testing (QT) and pre-processing (FIG. 5H). Whole cell (FIG. 5I), nucleus (FIG. 5J), and cytoplasm (FIG. 5K) average spectra of WT and HD SV40T CBL astrocytes. For visual purpose 2^{nd} derivative normalized spectra are displayed between 3050-2800 cm⁻¹ (lipid-rich region) and 1800-900 cm⁻¹ (“fingerprint” region).

FIGS. 6A-6F. Segmented cell spectra of striatum and cerebellum astrocytes. Whole cell, nucleus, and cytoplasm average spectra of WT and HD SV40T STR (FIGS. 6A-6C) and CTX (FIGS. 6D-6F) astrocytes. For visual purpose 2^{nd} derivative normalized spectra are displayed between 3050-2800 cm⁻¹ (lipid-rich region) and 1800-900 cm⁻¹ (“fingerprint” region).

FIGS. 7A-7J. Spectral phenotyping accurately predicts (or determines) disease class in HD astrocytes. UMAP clustering and classification derived from segmented whole cell (FIGS. 7A-7C), cytoplasm (FIGS. 7D-7F) or nucleus (FIGS. 7G-7I) for three regions of the brain CBL (FIGS. 7A, 7D, and 7G), STR (FIGS. 7B, 7E, and 7H) and CTX (FIGS. 7C, 7F, and 7I). FIG. 7J. Confusion matrices corresponding to each UMAP shown in FIGS. 7A-7I. The predicted and actual classification results for HD and WT astrocytes in the whole cell, cytoplasm, and nucleus for all three brain regions are listed in Table 1.

FIGS. 8A-8B. PCA clustering distinguishes HD from WT for the three brain regions as in FIGS. 7A-7J. FIG. 8A. PCA plots corresponding to the UMAP analysis for the three brain regions performed in FIGS. 4A-4F. FIG. 8B. PC1 (left) and PC2 (right) loading for the WT and HD samples from the CBL whole cell PCA (top left corner). PC loadings showed that lipid features (PC1 loading) and amide bands (PC2 loading) had a high contribution to the WT and HD cell discrimination.

FIGS. 9A-9C. Astrocytes have regional signatures that are distinguishable by their FTIR signatures. FIGS. 9A-9B. Pairwise classification of astrocytes isolated from the CBL, STR and CTX brain regions of SV40T WT (FIG. 9A) or HD (FIG. 9B) animals by UMAPs of 2^{nd} derivative normalized absorbance FTIR spectra (whole cells). FIG. 9C. Average 2^{nd} derivative normalized spectra of WT (left) and HD (right) SV40T astrocytes from the CBL (blue), STR (orange), CTX (green) brain regions. Spectra are displayed between 3050-2800 cm⁻¹ (lipid-rich region) and 1800-900 cm⁻¹ (“fingerprint” region). S, silhouette score (p-value: <0.001); A, accuracy.

FIGS. 10A-10K. FTIR substrates and coatings have an influence on cell spectra without altering disease/control classification. FIG. 10A. Experimental protocol schematic representing SV40T CTX WT or HD astrocytes cultured overnight on CaF₂ and Si substrates. Cells were fixed and dried prior to the FTIR acquisition. FIGS. 10B-10C. UMAP clustering results of WT (FIG. 10B) or HD (FIG. 10C) cells grown on CaF₂ and Si substrates. FIGS. 10D-10E. UMAP classification of WT and HD astrocytes grown on either CaF₂ (FIG. 10D) or Si (FIG. 10E) substrates. FIG. 10F. Schematic of substrate coating effect experiment following the same procedure as in FIG. 10. SV40T CTX WT or HD astrocytes were cultured overnight onto CaF₂ substrates uncoated (UN), with poly-L-ornithine dry (PLO-d) or poly-L-ornithine wet (PLO-w) coatings. FIGS. 10G-10H. UMAP clustering results for all three coatings on CaF₂ substrates for WT (FIG. 10G) or HD (FIG. 10H) cells. FIGS. 10I-10K. UMAP classification of WT and HD astrocytes grown on CaF₂ substrates uncoated (FIG. 10I) or coated with PLO-d (FIG. 10J) and PLO-w (FIG. 10K). All UMAP analyses were performed on 2^{nd} derivative normalized absorbance FTIR spectra of whole cells. S, silhouette score (p-value: <0.001); A, accuracy.

FIGS. 11A-11D. Best practice conditions for reproducibility of the FTIR signatures measured under various conditions. Reproducibility of cell spectra under various conditions was assessed by UMAP (left) and PCA (right) analysis. FIG. 11A. Technical replicates (TR) reproducibility. The S* and A* values were calculated for TR1 and TR5. FIG. 11B. Storage at RT. The S** and A** values are calculated for NS (no storage) and wk2. FIG. 11C. Storage at −80° C.; the S and A values are calculated for 5 days (d) and 5 months (m). FIG. 11D. Samples not stored (NS) compared to measurements after Freeze (−80° C.) and thaw (RT) cycles. The S*** and A*** values calculated for NS and FT4.

FIGS. 12A-12F. Spectral phenotyping can predict human neurodegenerative disease class from fibroblasts. FTIR spectra from human skin fibroblasts of controls (C) versus Huntington's disease (HD) (FIGS. 12A and 12B), controls (C) versus Alzheimer's disease (AD) (FIGS. 12C and 12D) or a comparison of HD and AD (FIGS. 12E and 12F) were evaluated by UMAP. The UMAP plots are the results of either pooled control or pooled disease samples (FIGS. 12A, 12C, and 12E), or displayed per individuals (FIGS. 12B, 12D, and 12F). All UMAP analyses were performed on 2^{nd} derivative normalized FTIR spectra of whole cells. S, silhouette score (p-value: <0.001); A, accuracy.

FIGS. 13A-13F. The PCA analysis corresponding to the UMAP analysis (FIGS. 12A-12F) for control and various disease fibroblast samples. FTIR spectra from human skin fibroblasts of controls (C) and Huntington's disease (HD) (FIGS. 13A and 13B), controls (C) and Alzheimer's disease (AD) (FIGS. 13C and 13D), and HD versus AD (FIGS. 13E and 13F) patients were evaluated by PCA. The PCA plots are the results of either pooled control or pooled disease samples (FIGS. 13A, 13C, and 13E), or displayed per individuals (FIGS. 13B, 13D, and 13). All PCA analyses were performed on 2^{nd} derivative normalized FTIR spectra of whole cells. S: silhouette score (p-value: <0.001), A: accuracy.

FIGS. 14A-14C. HD and AD spectral signatures. Mean second derivative normalized FTIR spectra (whole cells) of HD (FIG. 14A) and AD (FIG. 14B) from FIGS. 12A-12F and FIGS. 13A-13F, compared to the signature of control (C) cells. FIG. 14C. Direct comparison of the HD and AD spectral signatures. For visual purpose 2^{nd} derivative normalized spectra are displayed between 3050-2800 cm⁻¹ (lipid-rich region) and 1800-900 cm⁻¹ (“fingerprint” region).

FIGS. 15A-15C. FTIR discriminates among neurological disease. FIGS. 15A-15B. Representative PCA analysis of the FTIR signature spectra of human fragile X premutation (P, yellow in FIG. 15A) and control fibroblasts (green in FIG. 15A), as labeled. FIG. 15C. Combined plot of Fragile X premutation syndrome of premutation (P, yellow) and full mutation (F, red), compared to normal (NOR green) fibroblasts and to unrelated HD fibroblasts (blue), as disease groups (color coded). Fragile X is a systemic disease with neurological disease symptoms. It is generated from an expansion of repeating CGG in the intron of the FMR-1 gene: normal level is below 50 CGG repeats; premutation carriers (55-200) are susceptible to disease; full mutation is disease range is >200 repeats and expresses full disease phenotype.

FIGS. 16A-16D. FTIR discriminates among other disease that are not neurodegenerative. Representative PCA analysis of the FTIR signature spectra of (FIG. 16A) human normal epithelial cells and breast cancer epithelial cells; and (FIG. 16B) human Alzheimer's fibroblasts. Red is disease and green are control. FIG. 16C. Combined plot of Fragile X premutation syndrome of (P, premutation yellow), and (F, full mutation), compared to normal (NOR green) fibroblasts and to unrelated HD fibroblasts (blue), as disease groups (color coded). Fragile X is a systemic disease with neurological disease symptoms. It is generated from an expansion of repeating CGG in the intron of the FMR-1 gene: normal level is below 50 CGG repeats; premutation carriers (55-200) are susceptible to disease; full mutation is disease range is >200 repeats and expresses full disease phenotype. FIG. 16D. PCA of Fragile X patients and controls plotted as individuals. Each individual patient and control is color coded. Spectral phenotyping has applications for personalized medicine, although more detailed analysis will be needed to sort them discretely.

FIG. 17 is a block diagram of an illustrative computing system configured to implement any method of the present disclosure.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for determining a state of a test subject. In some embodiments, a method for determining a state of a test subject can be under control of a processor (e.g., a hardware processor or a virtual processor). The method can comprise: generating a plurality of reference Fourier transform infrared spectroscopy (FTIR) spectra (e.g., absorption spectra) for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples and the average test FTIR spectrum into a first cluster and a second cluster corresponding to the first state and the second state, respectively. The method can comprise: determining the test sample is in the first state or the second state based on whether the average test FTIR spectrum is in the first cluster or the second cluster.

Disclosed herein include methods for determining a state of a test subject. In some embodiments, a method for determining a state of a test subject is under control of a processor (e.g., a hardware processor or a virtual processor). The method can comprise: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples into a first cluster and a second cluster corresponding to the first state and the second state, respectively (e.g., in a reduced dimensionality space). The method can comprise: determining the test sample is in the first state or the second state based on a first distance between the average test FTIR spectrum and the first cluster and a second distance between the average test FTIR spectrum and the second cluster (e.g., in the reduced dimensionality space). The method can comprise: determining the test sample is in the first state or the second state based on the states of k-nearest neighbors of the average test FTIR spectrum (e.g., in the reduced dimensionality space).

Disclosed herein include embodiments of a computer readable medium. In some embodiments, a computer readable medium comprising executable instructions, when executed by a processor (e.g., a hardware processor or a virtual processor) of a computing system or a device, cause the processor, to perform any method disclosed herein.

Disclosed herein include systems for determining a state of a test subject. In some embodiments, a system for determining a state of a test subject comprises: non-transitory memory configured to store executable instructions. The system can comprise: a processor (e.g., a hardware processor or a virtual processor) in communication with the non-transitory memory, the hardware processor programmed by the executable instructions to perform: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The processor can be programmed by the executable instructions to perform: determining an average reference FTIR spectrum of the plurality of reference FTIR spectra for each of the plurality of reference samples. The processor can be programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples and the average test FTIR spectrum into a first cluster and a second cluster corresponding to the first state and the second state, respectively. The processor can be programmed by the executable instructions to perform: determining the test sample is in the first state or the second state based on whether the average test FTIR spectrum is in the first cluster or the second cluster.

Disclosed herein include systems for determining a state of a test subject. In some embodiments, a system for determining a state of a test subject comprises: non-transitory memory configured to store executable instructions and an average reference Fourier transform infrared spectroscopy (FTIR) spectrum of a plurality of reference FTIR spectra for each of a plurality of reference samples. The plurality of reference samples can comprise a plurality of first reference samples obtained from first reference subjects known to be in a first state and a plurality of second reference samples obtained from reference subjects known to be a second state. The system can comprise: a hardware processor in communication with the non-transitory memory, the hardware processor programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples and the average test FTIR spectrum into a first cluster and a second cluster corresponding to the first state and the second state, respectively. The processor can be programmed by the executable instructions to perform: determining the test sample is in the first state or the second state based on whether the average test FTIR spectrum is in the first cluster or the second cluster.

Disclosed herein include systems for determining a state of a test subject. In some embodiments, a system for determining a state of a test sample comprises: non-transitory memory configured to store executable instructions. The system can comprise: a hardware processor in communication with the non-transitory memory, the hardware processor programmed by the executable instructions to perform: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The processor can be programmed by the executable instructions to perform: determining an average reference FTIR spectrum of the plurality of reference FTIR spectra for each of the plurality of reference samples. The processor can be programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples into a first cluster and a second cluster corresponding to the first state and the second state, respectively, in a reduced dimensionality space. The processor can be programmed by the executable instructions to perform: determining the test subject is in the first state or the second state based on a first distance between the average test FTIR spectrum and the first cluster and a second distance between the average test FTIR spectrum and the second cluster.

Disclosed herein include systems for determining a state of a test sample. In some embodiments, a system for determining a state of a test sample comprises: non-transitory memory configured to store executable instructions and an average reference Fourier transform infrared spectroscopy (FTIR) spectrum of a plurality of reference FTIR spectra for each of a plurality of reference samples. The plurality of reference samples can comprise a plurality of first reference samples obtained from first reference subjects known to be in a first state and a plurality of second reference samples obtained from reference subjects known to be a second state. The system can comprise: a hardware processor in communication with the non-transitory memory, the hardware processor programmed by the executable instructions to perform: generating a plurality of test FTIR spectra for a test sample obtained from a test subject. One or more characteristics of the test subject and the reference subjects can be matched. The processor can be programmed by the executable instructions to perform: determining an average test FTIR spectrum of the plurality of test FTIR spectra for the test sample. The processor can be programmed by the executable instructions to perform: clustering the average reference FTIR spectra of the plurality of reference samples into a first cluster and a second cluster corresponding to the first state and the second state, respectively, in a reduced dimensionality space. The processor can be programmed by the executable instructions to perform: determining the test sample is in the first state or the second state based on a first distance between the average test FTIR spectrum and the first cluster and a second distance between the average test FTIR spectrum and the second cluster.

### Determining a State of a Test Subject

There is no access to the brain, during life, making a definitive diagnosis for neurologic or neurodegenerative disease difficult and precludes the opportunity to treat a patient. Disclosed herein include a reliable general-use biomarker to predict neurodegenerative disease in living patients using skin cells as surrogates. However, the method is applicable to any disease and has been used effectively in cases of breast cancer, Fragile X syndrome, among others. The general-use approach is based on infrared (IR) spectral imaging of cells to detect their chemical properties. Tens of thousands of chemical features in the skin cells are computationally integrated into a single “fingerprint” spectrum whose composition robustly characterizes each cell type or disease state. The wide availability of fibroblasts provides new opportunities to collect samples from living patients in any disease and create a reliable diagnostic tool that distinguish among disease subtypes, which are often misdiagnosed or are difficult to achieve using other methods. The applications apply broadly across disease type, to COVID infection detection, among others. Prediction uses accessible cell types, not only in skin, but also buccal cells (cheek swabs).

Cell Prediction. In some embodiments, after 7-10 days, skin cells are plated and cultured overnight onto IR compatible calcium fluoride (CaF2) substrates, fixed and dried before the spectral analysis. Brightfield imaging check on morphology followed by IR imaging.

Computational. In some embodiments, IR images are reconstructed on the amide I band (AI) for optimal background/cell contrast. Each tile can comprise 128 by 128 pixels (5.5 μm2), each of which contains a FTIR spectrum (in blue), thus constituting hyperspectral images. The raw spectral images can be carried through three processing steps to generate a cell signature. (Segmentation) The cells are segmented to extract from IR images the nucleus, cytoplasm, and whole cell raw spectra. (Pre-processing) Raw spectra are pre-processed to generate normalized second derivative spectra (Classification and statistics). Statistical analysis can be used to evaluate the disease classification using Principal Component Analysis (PCA) and Uniform Manifold Approximation and Projection (UMAP) analysis. Novel algorithm integrating computational methods: including Fourier transform, image segmentation, machine learning, watershed, baseline corrections, and statistics

The spectral phenotyping method of the disclosure can include one or more of the following properties: unique assembly of components; use of non tradition surrogate cells for disease predictions (e.g., skin cells to predict neurodegenerative disease or buccal cells); is applicable to accessible cell types, which can be collected easily without needing to access the disease tissue; non-traditional use of statistical methods; analysis is rapid (within an hour); prediction can accurately reflect disease status in cases where diagnosis is difficult or impossible using traditional methods. The method can be non-invasive, nondestructive, thus cells can be evaluated by IR light and used afterward for other testing; no a priori knowledge of the sample is needed.

The method can include the following steps:
Step 1. Obtain tissue sources for large cohorts of distinct diseases for FTIR analysis.
Step 2. Mining spectra for specific, fixed spectral parameters that uniformly classify among individual samples in the populations with high probability.
Step 3. Determine unique signatures for each disease, i.e., assign a spectrum identifier to each disease and build a knowledge-based repository for disease fingerprints.

Applications. The spectral phenotyping method of the present disclosure can aid in clinical diagnoses in living patients: many diseases are difficult to diagnose or are often confused with other disease (e.g. some forms of non-AD dementia are misclassified as Alzheimer's disease). An accurate classifier would be a significant advance and fill a large medical gap. The spectral phenotyping method of the present disclosure can be used in hospitals, clinical centers, private clinicians with practices, university-sponsored research applications, National Institutes of Health, Disease Foundations, pharmaceutical companies. The spectral phenotyping method of can be used for the development of therapeutics, as a rapid drug screening technology and/or following therapeutic treatment in patients during life: The FTIR disease signature can return to a normal fingerprint if treatment is successful.

The spectral genotyping method disclosed herein can include numerous advantages, such as speed: measurement are rapid versus other approaches; diagnosis can be successful after labor-intensive series of tests; FTIR is successful in hours. The use of surrogate cells for brain can be advantageous. Brain is not accessible during life but diagnosis is only important during life. An advantage can be accessibility: skin is accessible; collection is relatively non-invasive and can be collected from any patient. Additionally, the method can be used for therapeutic screening: reversal of the FTIR disease signature towards a normal spectra is a marker for therapeutic efficacy.

Disclosed herein include methods for determining a state of a test subject. In some embodiments, a method for determining a state of a test subject can be under control of a processor (e.g., a hardware processor or a virtual processor). The method can comprise: generating a plurality of reference Fourier transform infrared spectroscopy (FTIR) spectra (e.g., absorption spectra) for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more (e.g., 2, 3, 4, 5, 6, 7, 8 9, 10, or more) characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples and the average test FTIR spectrum into a first cluster and a second cluster corresponding to the first state and the second state, respectively. The method can comprise: determining the test sample is in the first state or the second state based on whether the average test FTIR spectrum is in the first cluster or the second cluster.

Disclosed herein include methods for determining a state of a test subject. In some embodiments, a method for determining a state of a test subject is under control of a processor (e.g., a hardware processor or a virtual processor). The method can comprise: generating a plurality of reference Fourier transform infrared spectroscopy (FTIR) spectra for each of a plurality of reference samples. The plurality of reference samples can comprise a plurality of first reference samples obtained from first reference subjects known to be in a first state and a plurality of second reference samples obtained from reference subjects known to be a second state. The method can comprise: determining an average reference FTIR spectrum of the plurality of reference FTIR spectra for each of the plurality of reference samples. The method can comprise: generating a plurality of test FTIR spectra for a test sample obtained from a test subject. One or more characteristics of the test subject and the reference subjects can be matched. The method can comprise: determining an average test FTIR spectrum of the plurality of test FTIR spectra for the test sample. The method can comprise: clustering the average reference FTIR spectra of the plurality of reference samples into a first cluster and a second cluster corresponding to the first state and the second state, respectively (e.g., in a reduced dimensionality space). The method can comprise: determining the test sample is in the first state or the second state based on a first distance between the average test FTIR spectrum and the first cluster and a second distance between the average test FTIR spectrum and the second cluster (e.g., in the reduced dimensionality space). The method can comprise: determining the test sample is in the first state or the second state based on the states of k-nearest neighbors of the average test FTIR spectrum (e.g., in the reduced dimensionality space).

In some embodiments, each of the plurality of reference samples and/or the test sample comprises, comprises about, comprises at least, comprises at least about, comprises at most, or comprises at most about, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values, cells. Each of the plurality of reference samples and the test sample can comprise about the same number of cells (e.g., within 1%, 2%, 3%, 4%, 5%, 6%, 7%, 8%, 9%, 10%, 15%, or 20%). In some embodiments, the sample comprises a tissue sample. The tissue sample can be, be about, be at least, be at least about, be at most, or be at most about, 5 m, 6 μm, 7 μm, 8 μm, 9 μm, 10 μm, 11 μm, 12 μm, 13 μm, 14 μm, 15 μm, 16 μm, 17 μm, 18 am, 19 μm, 20 μm, 25 μm, 30 μm, 40 μm, 50 μm, or a number or a range between any two of these values, in thickness. The tissue sample can comprise or comprise about one layer of cells. In some embodiments, the sample comprises surrogate cells (e.g., surrogate cells for neural cells, such as brain cells, or for cancer cells). The surrogate cells can comprise epithelial cells, fibroblasts, lymphoblasts, peripheral cells, non-neural cells, induced pluripotent stem cells, or a combination thereof.

In some embodiments, the plurality of reference samples and the test sample comprise fixed cells on slides. In some embodiments, the plurality of reference samples and the test sample were prepared in an identical manner. Preparation conditions of the plurality of reference samples and preparation conditions of the test sample were matched (e.g., in terms of the storage temperature, slide preparation and coating). In some embodiments, the slides comprise Calcium fluoride (CaF₂) or silicon (Si) slides. The slides can comprise no coating. The slides can comprise a coating. The coating can comprise poly-L-ornithine (PLO). The coating can comprise wet PLO or dry PLO. In some embodiments, the slides were previously stored at room temperature or −80° C. prior to the capturing of spectra. The slides may be previously stored at 40° C., 30° C., 20° C., 10° C., 0° C., −10° C., −20° C., −30° C., −40° C., −50° C., −60° C., −70° C., −80° C., or a number or a range between any two of these values, prior to the capturing of spectra. The duration of storage can be 1 day, 2 days, 3 days, 4 days, 5 days, 6 days 7 days, 2 weeks, 3 weeks, 4 weeks, 1 month, 2 months, 3 months, 4 months, 5 months, 6 months, or a number or a range between any two of these values. In some embodiments, the plurality of reference samples comprises, comprises at least, comprises at least about, comprises at most, or comprises at most about, 5, 10, 20, 30, 40, 50, 60, 70, 80, 90, 100, 200, 300, 400, 500, 600, 700, 800, 900, 1000, or a number or a range between any two of these values, samples. In some embodiments, the plurality of first reference samples comprises, comprises at least, comprises at least about, comprises at most, or comprises at most about, 5, 10, 20, 30, 40, 50, 60, 70, 80, 90, 100, 200, 300, 400, 500, 600, 700, 800, 900, 1000, or a number or a range between any two of these values, samples. In some embodiments, the plurality of second reference samples comprises, comprises at least, comprises at least about, comprises at most, or comprises at most about, 5, 10, 20, 30, 40, 50, 60, 70, 80, 90, 100, 200, 300, 400, 500, 600, 700, 800, 900, 1000, or a number or a range between any two of these values, samples.

In some embodiments, the plurality of reference FTIR spectra for each of the plurality of samples and the plurality of test FTIR spectra were captured in an identical manner. Capturing conditions of the plurality of reference FTIR spectra for each of the plurality of samples and capturing conditions the plurality of test FTIR spectra were matched (e.g., in terms of capturing temperature, capturing duration, capturing instrument, or IR intensity). In some embodiments, generating the plurality of reference FTIR spectra for each of the plurality of samples and the plurality of test FTIR spectra comprises capturing the plurality of reference FTIR spectra for each of the plurality of samples and the plurality of test FTIR spectra at room temperature or −80° C. Generating the plurality of reference FTIR spectra for each of the plurality of samples and the plurality of test FTIR spectra can comprise capturing the plurality of reference FTIR spectra for each of the plurality of samples and the plurality of test FTIR spectra at The slides may be previously stored at 40° C., 30° C., 20° C., 10° C., 0° C., −10° C., −20° C., −30° C., −40° C., −50° C., −60° C., −70° C., −80° C., or a number or a range between any two of these values.

In some embodiments, the first state comprises a first phenotype (e.g., non-diseased or non-responsive), and the second state comprises a second phenotype (e.g., diseased or responsiveness). The first state can be non-responsiveness to a treatment of a disease, and the second state can be responsiveness to the treatment of the disease. The first state can be a non-diseased state, and the second state can be a diseased state. The disease can be a disease subtype. The disease can be a disease of the brain. The disease can be a neurological disease, a neurodegenerative disease, a late onset disease, or a cancer. The neurological disease or the neurodegenerative disease can comprise Alzheimer's disease, Huntington's disease, or Fragile X syndrome. The disease (or phenotype, or state) can be Alzheimer's Disease, Huntingon Disease, Exected-Brain, Parkinson's disease, Motor neuron disease, Multiple system atrophy, Progressive supranuclear palsy, Multiple sclerosis. The disease (or phenotype, or state) can be Autism Spectrum, Schizophrenia, Acute Spinal Cord Injury, Alzheimer's Disease, Amyotrophic Lateral Sclerosis (ALS), Ataxia, Bell's Palsy, Brain Tumors, Cerebral Aneurysm, Epilepsy and Seizures, Guillain-Barre Syndrome, Headache, Head Injury, Hydrocephalus, Lumbar Disk Disease (Herniated Disk), Meningitis, Multiple Sclerosis, Muscular Dystrophy, Neurocutaneous Syndromes, Parkinson's Disease, Stroke (Brain Attack), Cluster Headaches, Tension Headaches, Migraine Headaches, Encephalitis, Septicemia, Types of Muscular Dystrophy and Neuromuscular Diseases, Myasthenia Gravis, Gliomas, Nueroblastomas, and Stroke. The method can be used for diagnosing, treatment monitoring, and/or rehabilitation of a disease (or phenotype, or state).

A cancer can be melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and Multiple myeloma (“MM”).

The cancer can be renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

In some embodiments, the one or more characteristics of the test subject and the reference subjects that are matched comprise age, gender, lifestyle, diet, health, ethnicity, and/or medical background (e.g., cholesterol level). In some embodiments, the second reference subjects have no symptoms, have no overt symptoms, is pre-symptomatic, and/or is pre-disease onset.

In some embodiments, the plurality of reference FTIR spectra, the average reference FTIR spectra, the plurality of test FTIR spectra, and the average test FTIR spectra comprise second derivative absorbance spectra. In some embodiments, the plurality of reference FTIR spectra and/or the plurality of test FTIR spectra comprises 100, 200, 300, 400, 500, 600, 700, 800, 900, 1000, 2000, 3000, 4000, 5000, 6000, 7000, 8000, 9000, 10000, or a number or a range between any two of these values, spectra. In some embodiments, the plurality of reference FTIR spectra, the average reference FTIR spectra, the plurality of test FTIR spectra, and the average test FTIR spectra comprise spectra between 3050-2800 cm⁻¹ and/or 1800-900 cm⁻¹. A spectrum can include one continuous spectrum. A spectrum can include one or more discontinuous subspectra. The upper bound of a spectrum or a subspectrum can be, be about, be at least, be at least about, be at most, or be at most about, 3300 cm⁻¹, 3250 cm⁻¹, 3200 cm⁻¹, 3150 cm⁻¹, 3100 cm⁻¹, 3050 cm⁻¹, 3000 cm⁻¹, 2950 cm⁻¹, 2900 cm⁻¹, 2850 cm⁻¹, 2800 cm⁻¹, 2750 cm⁻¹, 2700 cm⁻¹, 2650 cm⁻¹, 2600 cm⁻¹, 2550 cm⁻¹, 2500 cm⁻¹, 2450 cm⁻¹, 2400 cm⁻¹, 2350 cm⁻¹, 2300 cm⁻¹, 2250 cm⁻¹, 2200 cm⁻¹, 2150 cm⁻¹, 2100 cm⁻¹, 2050 cm⁻¹, 2000 cm⁻¹, 1950 cm⁻¹, 1900 cm⁻¹, 1850 cm⁻¹, 1800 cm⁻¹, 1750 cm⁻¹, 1700 cm⁻¹, 1650 cm⁻¹, 1600 cm⁻¹, 1550 cm⁻¹, 1500 cm⁻¹, 1450 cm⁻¹, 1400 cm⁻¹, 1350 cm⁻¹, 1300 cm⁻¹, 1250 cm⁻¹, 1200 cm⁻¹, 1150 cm⁻¹, 1100 cm⁻¹, 1050 cm⁻¹, 1000 cm⁻¹, or a number or a range between any two of these values. The lower bound of a spectrum or a subspectrum can be, be about, be at least, be at least about, be at most, or be at most about, 3250 cm⁻¹, 3200 cm⁻¹, 3150 cm⁻¹, 3100 cm⁻¹, 3050 cm⁻¹, 3000 cm⁻¹, 2950 cm⁻¹, 2900 cm⁻¹, 2850 cm⁻¹, 2800 cm⁻¹, 2750 cm⁻¹, 2700 cm⁻¹, 2650 cm⁻¹, 2600 cm⁻¹, 2550 cm⁻¹, 2500 cm⁻¹, 2450 cm⁻¹, 2400 cm⁻¹, 2350 cm⁻¹, 2300 cm⁻¹, 2250 cm⁻¹, 2200 cm⁻¹, 2150 cm-1, 2100 cm⁻¹, 2050 cm⁻¹, 2000 cm⁻¹, 1950 cm⁻¹, 1900 cm⁻¹, 1850 cm⁻¹, 1800 cm⁻¹, 1750 cm⁻¹, 1700 cm⁻¹, 1650 cm⁻¹, 1600 cm⁻¹, 1550 cm⁻¹, 1500 cm⁻¹, 1450 cm⁻¹, 1400 cm⁻¹, 1350 cm⁻¹, 1300 cm⁻¹, 1250 cm⁻¹, 1200 cm⁻¹, 1150 cm⁻¹, 1100 cm⁻¹, 1050 cm⁻¹, 1000 cm⁻¹, 950 cm⁻¹, 900 cm⁻¹, 850 cm⁻¹, 800 cm⁻¹, 750 cm⁻¹, 700 cm⁻¹, or a number or a range between any two of these values.

In some embodiments, the plurality of reference FTIR spectra for each of the plurality of reference samples and the plurality of test FTIR spectra comprise FTIR spectra generated from whole cells. In some embodiments, the plurality of reference FTIR spectra for each of the plurality of reference samples and the plurality of test FTIR spectra comprise FTIR spectra generated from cytoplasm of cells. In some embodiments, the method comprises segmenting (e.g., seed watershed segmentation) the plurality of reference FTIR spectra for each of the plurality of reference samples and the plurality of test FTIR spectra to determine reference FTIR spectra of the plurality of reference FTIR spectra for each of the plurality of reference samples and test FTIR spectra of the plurality FTIR spectra generated from cytoplasm of cells. The segmenting can be based on integrated absorbance frequencies between 1670-1630 cm⁻¹.

In some embodiments, the method comprises quality testing (e.g., to control for absorbance (A), signal to noise ratio (SNR), and signal to water vapor ratio (SWR)) the plurality of reference FTIR spectra for each of the plurality of samples and the plurality of test FTIR spectra to generate a plurality of quality-tested, reference FTIR spectra for each of the plurality of samples and the plurality of quality-tested, test FTIR spectra. The plurality of quality-tested reference FTIR spectra can include, include about, include at least, include at least about, include at most, or include at most about, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or a number or a range between any two of these values, of reference FTIR spectra of the plurality of reference FTIR spectra. The plurality of quality-tested test FTIR spectra can include, include about, include at least, include at least about, include at most, or include at most about, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or a number or a range between any two of these values, of test FTIR spectra of the plurality of test FTIR spectra.

Determining the average reference FTIR spectrum of the plurality of reference FTIR spectra for each of the plurality of reference samples can comprise determining an average reference FTIR spectrum of the plurality of quality-tested, reference FTIR spectra for each of the plurality of reference samples. Determining the average test FTIR spectrum can comprise determining the average test FTIR spectrum of the plurality of quality-tested, test FTIR spectra.

In some embodiments, the method comprises pre-processing the plurality of reference FTIR spectra for each of the plurality of samples and the plurality of test FTIR spectra to generate a plurality of pre-processed, reference FTIR spectra for each of the plurality of samples and the plurality of pre-processed, test FTIR spectra. The plurality of pre-processed reference FTIR spectra can include, include about, include at least, include at least about, include at most, or include at most about, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or a number or a range between any two of these values, of reference FTIR spectra of the plurality of reference FTIR spectra (or quality-tested reference FTIR spectra of the plurality of quality-tested reference FTIR spectra). The plurality of pre-processed test FTIR spectra can include, include about, include at least, include at least about, include at most, or include at most about, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or a number or a range between any two of these values, of test FTIR spectra of the plurality of test FTIR spectra (or quality-tested test FTIR spectra of the plurality of quality-tested test FTIR spectra).

Determining the average reference FTIR spectrum of the plurality of reference FTIR spectra for each of the plurality of reference samples can comprise determining an average reference FTIR spectrum of the plurality of pre-processed, reference FTIR spectra for each of the plurality of reference samples. Determining the average test FTIR spectrum can comprise determining the average test FTIR spectrum of the plurality of pre-processed, test FTIR spectra. Pre-processing can comprise smoothing (e.g., using the Savitzky-Golay method), baseline correction, spectral contrast optimization, and/or vector normalization.

In some embodiments, the plurality of reference FTIR spectra for each of the plurality of reference samples and the plurality of test FTIR spectra comprise normalized second derivative spectra. In some embodiments, clustering the average reference FTIR spectra of the plurality of reference samples comprises dimensionality reduction. Clustering the average reference FTIR spectra of the plurality of reference samples can comprise unsupervised clustering. The unsupervised clustering comprises Principal Component Analysis (PCA) and Uniform Manifold Approximation and Projection (UMAP) analysis.

In some embodiments, a Silhouette score of the test sample being determined to be in the first state or the second state is, is about, is at least, is at least about, is at most, or is at most about, 0.3, 0.35, 0.4, 0.45, 0.5, 0.55, 0.6, 0.65, 0.7, 0.75, 0.8, 0.85, 0.9, 0.95, or a number or a range between any two of these values. Sensitivity of the test sample being determined to be in the first state or the second state can be, be about, be at least, be at least about, be at most, or be at most about, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, or a number or a range between any two of these values. Specificity of the test sample being determined to be in the first state or the second state can be, be about, be at least, be at least about, be at most, or be at most about, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, or a number or a range between any two of these values. Accuracy of the test sample being determined to be in the first state or the second state can be, be about, be at least, be at least about, be at most, or be at most about, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, or a number or a range between any two of these values.

In some embodiments, the average test FTIR spectrum is in the first cluster if a first distance between the average test FTIR spectrum and the first cluster is shorter than a second distance between the average test FTIR spectrum and the second cluster. The average test FTIR spectrum is in the first cluster if a first distance between the average test FTIR spectrum and the first cluster is longer than a second distance between the average test FTIR spectrum and the second cluster.

In some embodiments, the first distance between the average test FTIR spectrum and the first cluster comprises the first distance between the average test FTIR spectrum and a center of the first cluster. The second distance between the average test FTIR spectrum and the second cluster can comprise the second distance between the average test FTIR spectrum and a center of the second cluster. In some embodiments, the first distance between the average test FTIR spectrum and the first cluster comprises the first distance between the average test FTIR spectrum and k-nearest neighbors of the first cluster. The second distance between the average test FTIR spectrum and the second cluster comprises the second distance between the average test FTIR spectrum and k-nearest neighbor of the second cluster. k can be, be about, be at least, be at least about, be at most, be at most about, 3, 4, 5, 6, 7, 8, 9, 10, 15, 20, 30, 40, 50, 60, 70, 80, 90, 100, or a number or a range between any two of these values.

### EXAMPLES

Some aspects of the embodiments discussed above are disclosed in further detail in the following examples, which are not in any way intended to limit the scope of the present disclosure.

### Example 1

### An Infrared Spectral Biomarker Accurately Predicts Neurodegenerative Disease Class in the Absence of Overt Symptoms

### Overview

Although some neurodegenerative diseases can be identified by behavioral characteristics relatively late in disease progression, there is currently no methods to predict (or determine) who has developed or develop a disease before the onset of symptoms, when onset will occur, or the outcome of therapeutics. New biomarkers are needed. This example describes spectral phenotyping, a new kind of biomarker that makes disease predictions based on chemical rather than biological endpoints in cells. Spectral phenotyping uses Fourier transform infrared (FTIR) spectromicroscopy to produce an absorbance signature as a rapid physiological indicator of disease state. This example describes the unique FTIR chemical signature can accurately predict disease class in mouse with high probability in the absence of brain pathology. In human cells, the FTIR biomarker can accurately predict (or determine) neurodegenerative disease class using fibroblasts as surrogate cells.

### Introduction

Although some disease-causing mutations are well known, the vast amount of available data have not necessarily led to robust disease detection, or to a good understanding of disease etiology, particularly for neurodegeneration. The identification of reliable disease biomarkers has been difficult and hindered by the fact that the brain is not an accessible tissue. Thus, classification relies on clinical diagnosis, which is not always certain. Alzheimer's disease (AD) and Huntington's disease (HD) provide good examples. HD and AD are typically late onset diseases, which arise from neuronal loss in the striatum and hippocampus, respectively. However, the former is a dominant single-gene defect, while the underlying genetic causes of the latter are unknown for 95% of patients. There is no way to predict in advance who will develop AD or its onset. Moreover, the characteristic cognitive decline is not unique to AD and can occur during normal aging. Although a battery of neuropsychological tests is often used in making a clinical diagnosis of AD, a definitive diagnosis still relies on pathological evaluation of plaques and tangles at autopsy.

HD is characterized by motor decline, striatal death with well-defined genetics. The underlying mutation in HD is expansion of a CAG triplet repeat tract in exon 1 of the expressed disease allele. Using traditional genetic screens, the onset of HD is predictable by the length of the CAG repeat tract. The longer the tract, the more severe is the phenotype. However, there are unknown modifier genes whose effects vary with the patient. While the onset of HD patients with a CAG tract of 50 is on the average around 50 years of age, the onset of any particular patient with a repeat tract length of 50 can vary as much as 4-fold, ranging from 20 to 80 years of age. Thus, quality of life can differ significantly among HD patients of the same repeat tract length, but disease outlook is not always certain. The pathology in a brain section is obvious for an HD or an AD patient after death, and biomarkers are not needed to make a postmortem diagnosis. However, an early biomarker to predict disease during life would be a significant advance.

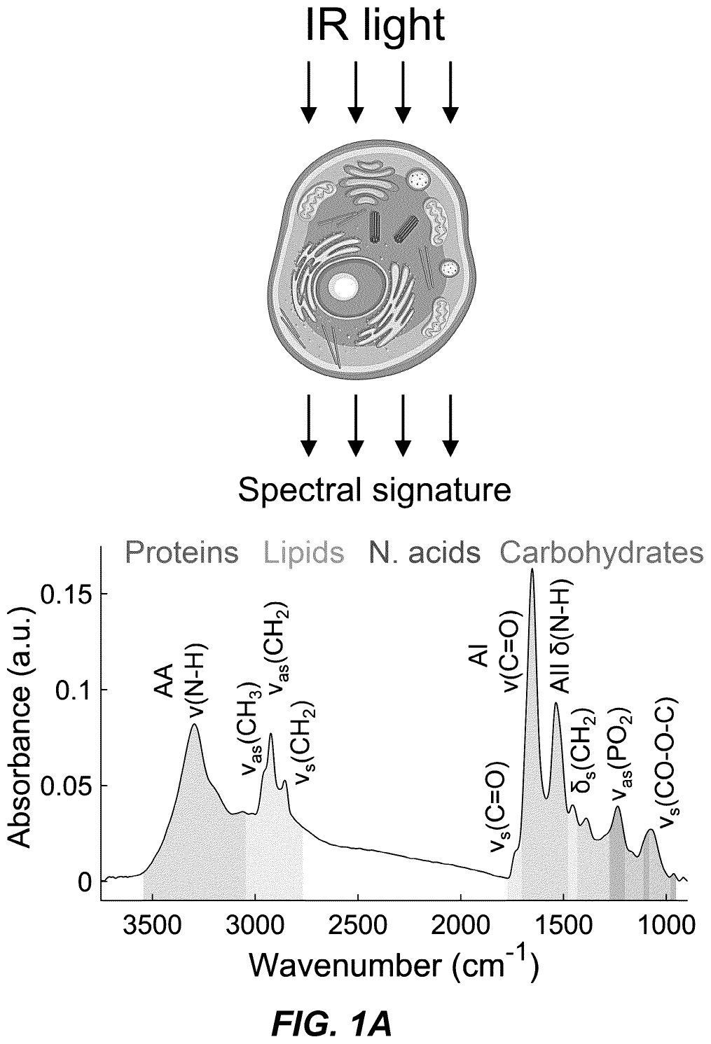

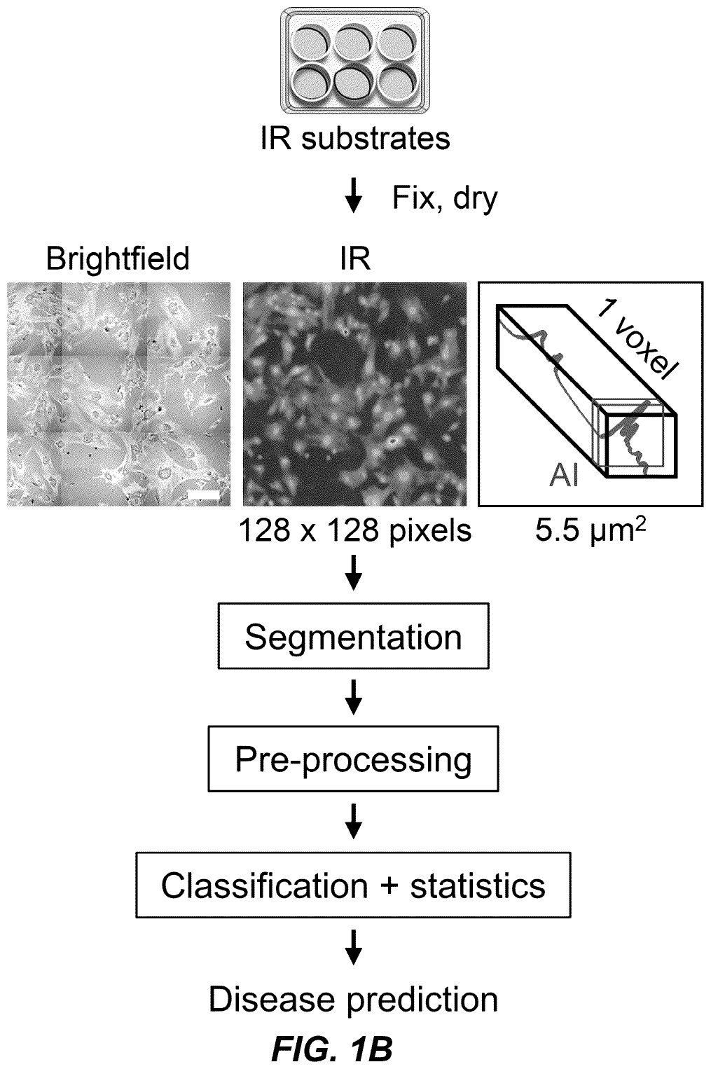

Towards this effort, this example describes a general-use Fourier transform infrared (FTIR) technology which predicts disease class with high probability. Over the years, FTIR as well as Raman microspectroscopies have emerged as useful tools for characterization of biological samples based on their unique chemistry and spectral properties (FIG. 1A). Indeed, infrared irradiation produces an absorbance spectrum that integrates the vibrational state of tens of thousands of endogenous chemical features (FIG. 1A). The resulting absorbance spectrum does not correspond to a single molecule. Rather, it is an integrated physiological “read-out” of all molecular bonds originating from the function groups in proteins, lipids, carbohydrates, and nucleic acids. While all cells have the same collection of functional groups, band intensity and position will vary depending on the group's abundance, hydrogen bonding, bond angle, and molecular context. Thus, the composition of the FTIR signature fingerprints cells (FIG. 1A). The FTIR absorbance profile is a powerful discriminator since the profile is based on whole-cell chemistry rather than on specific biological endpoints or single point markers. Thus, the change in an FTIR absorbance spectrum reflects real physiological changes such as those that accompany a disease.

Based on its chemical richness, FTIR has been used successfully for differential diagnosis of cancer subtypes in patients with manifest disease, attesting to its powerful discrimination capability. However, the approach of this example goes further and shows that (1) a spectral phenotyping approach is capable of robust classification of neurodegenerative disease before the manifestation of overt symptoms in a mouse astrocyte model, and (2) disease prediction (or determination) is possible using non-neuronal human cells as surrogates. These are important capabilities since the human brain is not accessible during life and biological symptoms may occur too late in patients for effective therapeutics.

This example describes the development of spectral phenotyping, a reliable algorithm to predict (or determine) disease and non-disease classes. Both a standardized analytical approach and best practice metrics are critical parameters and are described for the analysis. The strategy followed a two-step plan: (1) to develop a robust algorithm using a stable mouse system with little biological variation, and (2) to test the prediction algorithm with more variable human HD or AD fibroblasts, which were used as brain cell surrogates. For the mouse experiments, the FTIR biomarker was benchmarked using a well characterized HdhQ(150/150) inbred model of HD and compared to its genetically matched control strain, C57Black6 (C57Bl6J), which do not express the mutant gene. The HdhQ(150/150) line harbors an expanded CAG repeat tract of 150 knocked into the endogenous mouse Huntington gene locus42. The HdhQ(150/150) line is a good model for “late onset” disease, since these animals express the mutant huntingtin (mhtt) disease protein at physiological levels from birth but do not display symptoms until late in life. Thus, HD animals from 2 days to 2 years were tested to assess the likelihood that an early disease prediction (or determination) by FTIR spectroscopy was possible in the absence of a disease phenotype. Spectral phenotyping was not only successful in disease classification in the absence of overt pathology in the mouse model, but also predicted neurodegenerative disease class in HD and AD patients using fibroblasts as surrogates for brain cells Results

FTIR signatures were acquired by mid-IR range light (wavelengths from 2.5 μm to 25 μm)²⁶⁻²⁸ and measuring the absorbance profile of vibrational frequencies (wavenumbers in cm⁻¹) between 4000 cm⁻¹ and 900 cm⁻¹ (FIG. 1A). The astrocytes were cultured on IR transparent calcium fluoride substrates (FIG. 1), and a user-defined number of adjacent field of views (FOV) were exposed to IR light. Their IR absorption spectra were collected at multiple wavelengths using a focal plane array (FPA) light detector, which is placed in the image plane of the microscope (FIG. 1). Within the 128 by 128 pixel FOV, each pixel (set to 5.5 μm²) of the hyperspectral image contained a complete FTIR absorbance spectrum (FIG. 1), which was processed to obtain the chemical signature for the cells. The steps of sample preparation, FTIR acquisition, image segmentation, analysis, and statistical pipeline (FIG. 1B) are briefly discussed in the results section, and the details are provided in the methods section.

FIGS. 1A-1B. Concept of cell phenotyping by infrared spectroscopy. FIG. 1A. Schematic of a representative infrared spectrum of astrocytes and the attribution of the prominent chemical features between 4000-900 cm⁻¹. AA/I/II: amide A/I/II, v: stretching, δ: bending, as: asymmetric, s: symmetric vibrations. FIG. 1B. Brief outline of the analysis pipeline for spectral phenotyping, as discussed in example 1. After 7-10 days, cells were plated and cultured overnight onto IR compatible calcium fluoride (CaF₂) substrates, fixed and dried before the spectral analysis. A representative brightfield and corresponding IR image of astrocytes are displayed. IR images were reconstructed on the amide I band (AI) for optimal background/cell contrast. Each tile comprises 128 by 128 pixels (5.5 μm²), each of which contains a FTIR spectrum (in blue), thus constituting hyperspectral images. The raw spectral images were carried through three processing steps to generate a cell signature. (Segmentation) The cells were segmented to extract from IR images the nucleus, cytoplasm, and whole cell raw spectra. (Pre-processing) Raw spectra were pre-processed to generate normalized second derivative spectra (Classification and statistics). Statistical analysis was used to evaluate the disease classification using Principal Component Analysis (PCA) and Uniform Manifold Approximation and Projection (UMAP) analysis. Scale bar=100 μm.

Early Postnatal Astrocytes from WT and HD Mice are Indistinguishable by Obvious Criteria.

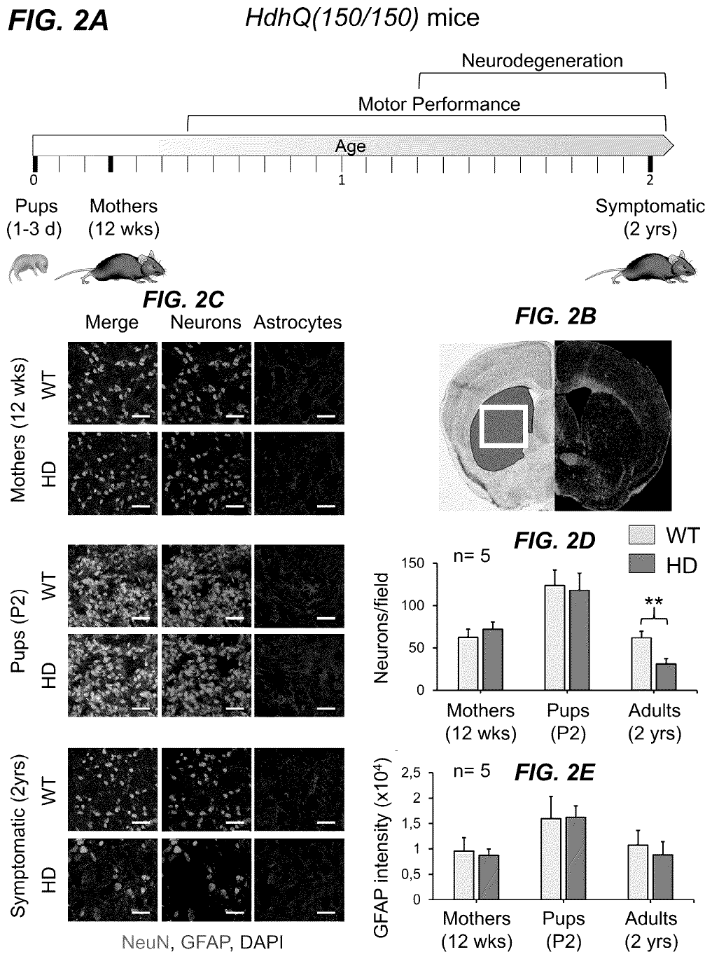

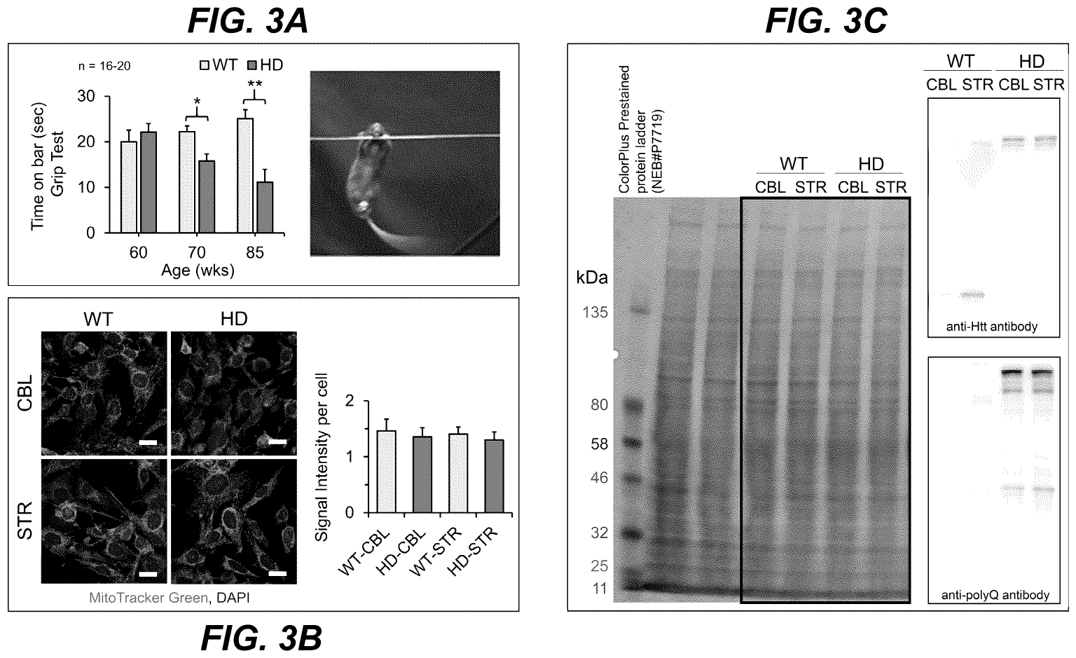

Spectral phenotyping was implemented for robust disease predictions in astrocytes isolated from C57Bl6J or HdhQ(150/150) animals, which are referred to as wild-type (WT) and HD, respectively. HD pathology was evaluated in brain sections from newborn pups at postnatal day 1-3 (referred to as P2) (FIG. 2A), in 12-week mothers, and in 2 year affected animals to establish the earliest non-symptomatic age window for FTIR analysis. The brains of the P2 pups displayed no obvious pathology (FIGS. 2C-2E). Indeed, pups of both genotypes had a similar number of neurons in the striatum (FIG. 2B), the region most prone to neural death in HD. As quantified by the NeuN antibody marker for neurons (FIGS. 2C and 2D) and the astroglial marker, Glial Fibrillary Acidic Protein (GFAP) (FIGS. 2C and 2E), there were no measurable changes in cell morphology or number in the brains of P2 animals. Similarly, the 12-week mothers also showed no brain pathology (FIGS. 2C-2E) and were asymptomatic by standard measures of motor function compared to WT animals of similar ages (FIG. 2A, FIG. 3A). This was in contrast to 2-year HD animals, which had lost roughly 50% of their neurons relative to WT animals of comparable age (FIGS. 2C and 2D) and had developed substantial motor dysfunction (FIG. 2A, FIG. 3A). Collectively, WT and HD P2 pups differed in genotype but were not distinguishable by overt phenotypes.

FIG. 3A. Grip test for motor function. The time in seconds is a measure of duration for gripping the bar (right). Performance is plotted as time (sec) versus age (wks) in WT and HD animals (left). The WT and HD animals had similar grip performance up to 60 weeks. n=16; * p-value: <0.05; ** p-value: <0.005 (Student's t-test, 2 tailed, equal variance homoscedastic).

FIGS. 2A-2E. HD mothers and their pups display no overt pathology relative to WT animals. FIG. 2A. Schematic summary of behavior in HdhQ(150/150) animals with age. The P2 pups, their mothers (12 weeks), and symptomatic 2-year animals are displayed on the timeline. FIG. 2B. Cartoon depicting an adult striatum in red and the white box indicating the regions probed in the brain slices in FIG. 2C. FIG. 2C. Mouse striatal brain sections were analyzed for neurons (NeuN antibody) alone, astrocytes (GFAP antibody) alone or as a merged image (Merge) of the two. The striatal regions were compared between WT and HD animals at various ages. There were no differences in neuronal counts in the striatum of HD animals compared to WT, except at very late ages (2 years of age). There was no significant difference in astrocyte levels (GFAP intensity per field) between HD and WT at any age. Scale bar is 50 μm. FIGS. 2D-2E. Quantification of neuronal counts and astrocyte counts from FIG. 2C. ** p-value:<0.005 (Student's t-test, 2 tailed, equal variance homoscedastic).

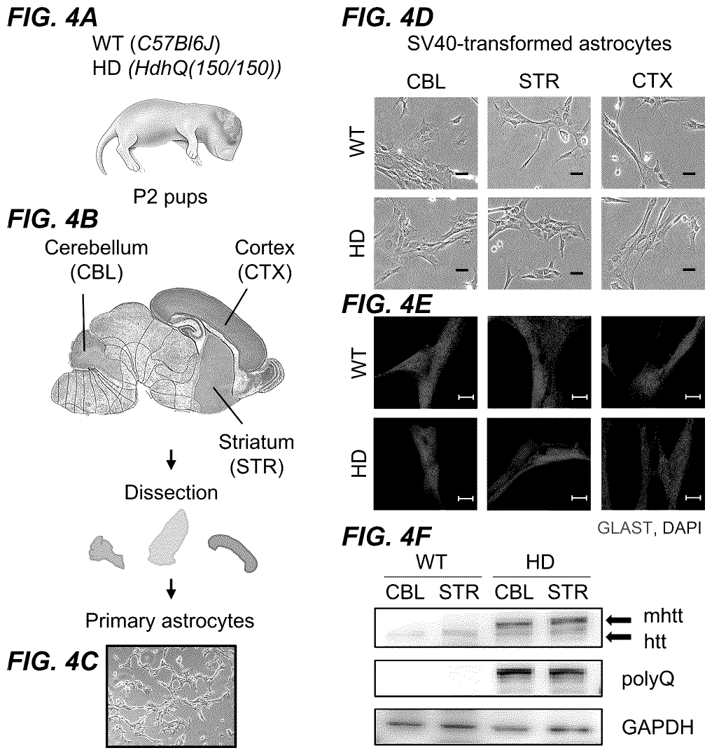

Astrocytes were purified from P2 pups. Whether cells from WT and HD animals could be distinguished by visual cues in culture were evaluated. FIGS. 4A and 4B show cartoons highlighting the three brain regions dissected for preparation of astrocytes; the striatum (STR) is the most susceptible region, the cortex (CTX), and the cerebellum (CBL), which is most resistant to neurodegeneration (FIGS. 4A and 4B). After dissection, the isolated astrocytes from each region (FIG. 4C) were immortalized with simian virus large T antigen (SV40T), as described in the methods section. The transformed cells provided clonally derived, continuous astrocyte lines to minimize batch effects. The WT and HD cells in culture were indistinguishable. The WT and HD cells had similar morphology as illustrated by the bright field (FIG. 4D) or immunofluorescence images (FIG. 4E) and had an equivalent number and activity of mitochondria, which were reflected in the intensity of Mitotracker Green signal (FIG. 3B). Indeed, there were no region-specific differences that were obvious by eye in any of the lines and all stained positively for Glutamate Aspartate Transporter 1 (GLAST1) (FIG. 4E), establishing their identity as astrocytes. Although the astrocyte cell lines from WT and HD animals retained expression of the huntingtin (htt) or mhtt protein, respectively (FIG. 4F, shown are CBL and STR: FIG. 3C), there were no physical cues to classify these cells as normal or disease. Thus, whether their chemistry, as judged by the FTTR spectral signature, could accurately predict the disease class of these astrocytes isolated at presymptomatic stages was tested.

FIGS. 4A-4F. Astrocyte cultures from WT and HD animals are visually indistinguishable. FIG. 4A. Astrocyte cell lines from CBL, STR, CTX were dissociated and isolated from the brains of postnatal (P2) mice, from either WT or HD mice. FIG. 4B. Cartoon showing the developing mouse brain at P4 and the dissected regions used in the analysis. The regions are schematically illustrated is the Nissl-stained brain image (purple) from P4 animals. FIG. 4C. A representative brightfield image of primary astrocytes from the cortex of WT mice. FIG. 4D. Purified SV40T astrocytes in all 3 brain regions from WT and HD mice. Scale bars=20 μm. FIG. 4E. Transformed cultures were stained for Glutamate Aspartate Transporter 1 (GLAST1) antibody marker to confirm their identity as astrocytes, as well as stained with DAPI to define the nucleus. Scale bars=20 μm. Cell lines of either genotype had similar morphology. FIG. 4F. Western blot analysis showing that mouse astrocytes from WT and HD mice express normal htt and the mutant (mhtt), respectively, in the STR and CBL. HD astrocytes alone express mhtt, which includes an expanded polyQ stretch. The loading control is total protein visualized with No-Stain Protein Labelling Reagent. The uncropped images are shown in FIG. 3C.

FIG. 3B. (left) Fluorescence staining of astrocytes with Mitotracker Green (green) to visualize mitochondria number and activity, which were equivalent in WT and HD cells. DAPI staining (blue) indicates the position of the nucleus. To the right is quantification of mitochondrial staining in astrocyte cultures from the CBL or the STR, as indicated. Light gray is WT and dark gray is HD; n=50 (right). Variance is reported as standard error. The scale bar is 10 μm. FIG. 3C. Full length uncropped western gels of normal and mutant huntingtin protein corresponding to the cropped images in FIG. 4F. (Left) Total protein loading control for the WT and HD animals in the cerebellum (CBL) and striatum (STR), as indicated, visualized with No-Stain Protein Labelling Reagent (Thermofisher). The boxed region corresponds to the four lanes in the gels on the right. (Right) The nitrocellulose blots were probed with an anti-Htt antibody (upper blot), to the normal huntingtin protein in the WT or to the faster migrating band in the heterozygous HD sample. The anti-polyQ antibody (lower blot) primarily detects the mutant protein in the slower migrating band in the HD sample.

### Cell Segmentation Increased the Accuracy of Predictions.

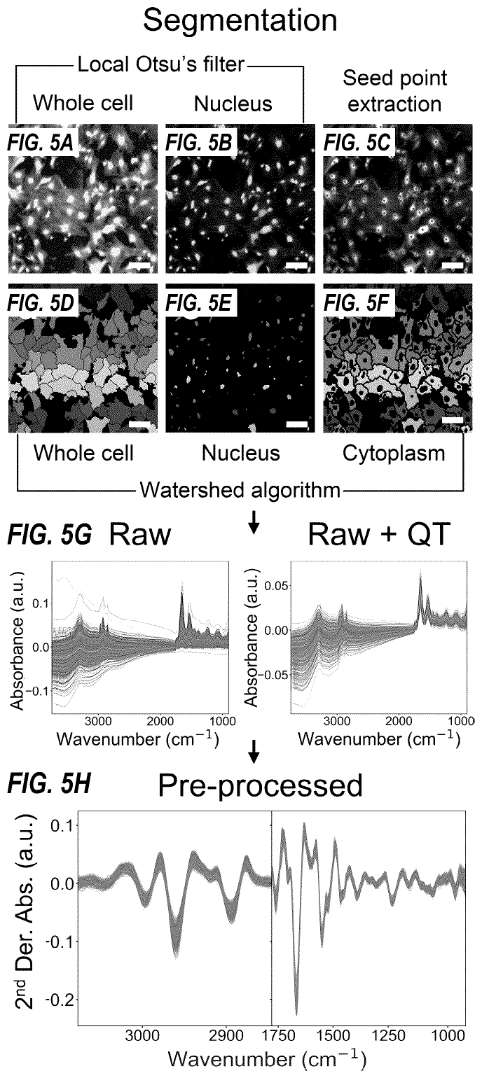

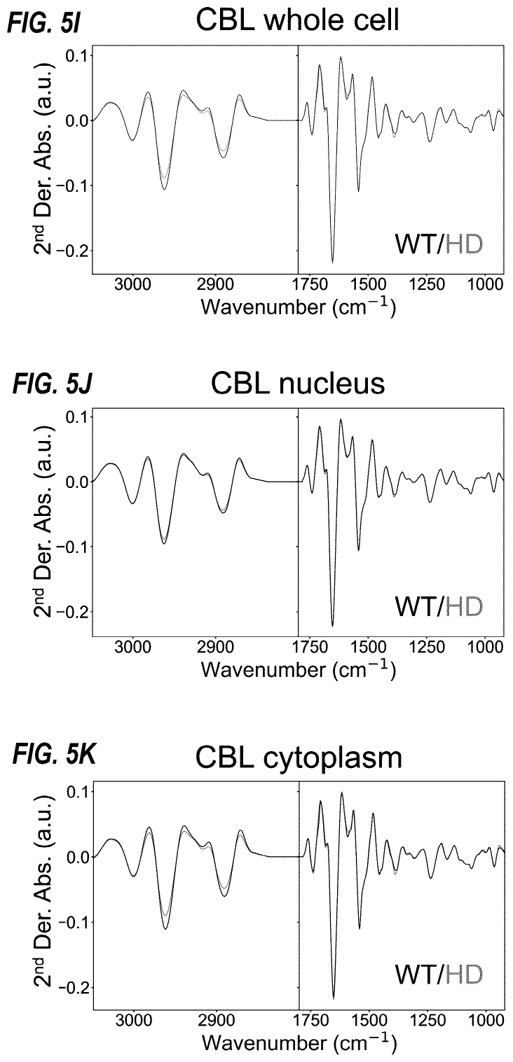

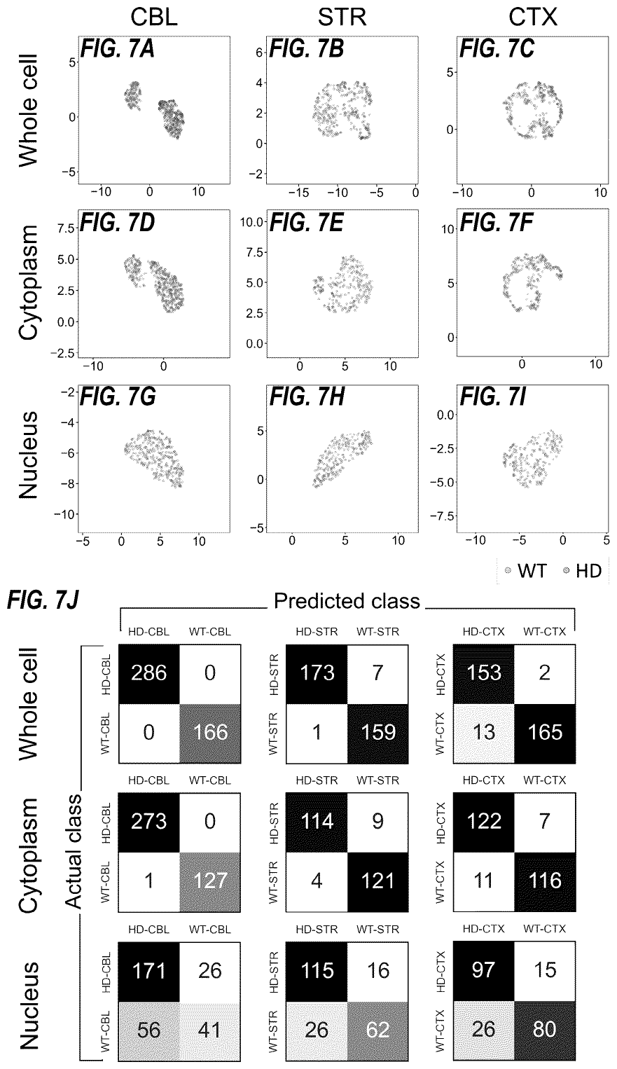

Spectral phenotyping can discriminate between WT and HD samples if their mean absorbance spectra differ. FTIR class is defined as disease (HD) or non-disease (WT). Thus, a robust disease prediction depends on the chemical features that contribute most to the differences (FIG. 1A). Because those features are not known apriori, whether cell segmentation would identify a best subcellular site for spectral acquisition was considered. For example, the high contrast of the nucleus is a desirable segment to extract discriminant IR or Raman spectral features. However, if features of the cytosol provided a major contribution to the spectral differences, then the nuclear segment might not be ideal for disease predictions. The hyperspectral images were segmented (FIGS. 5A-5F) using the Otsu's algorithm (FIGS. 5A-5B) followed by the seed point-watershed algorithm (FIGS. 5C-5F). The cell segmentation was performed before the spectral pre-processing. Thus, the signatures from each segment were based on the integrated absorbance frequencies between 1670-1630 cm⁻¹ (amide I band) for each pixel, and not on biochemical differences. Nonetheless, the (absorbance) difference between cytoplasm and condensed matter of the nucleus is large and the signatures derived from the whole cell, the cytoplasm and the nuclear segments were distinct in the WT and HD comparison (FIGS. 7A-7J). The segmentation approach enabled a fast, semi-automated distinction between nuclear and cytoplasmic segments in the image relative to the whole cell (FIGS. 5A-5F). Pixels that were designated as nuclei (FIG. 5E) were estimated from the maximum intensity variation between the image background and foreground, where foreground was defined as the cell center and the background is the whole cell (FIG. 5B). The pixels, which were designated as the cytoplasm (FIG. 5F), were derived by subtracting the pixels designated as the nuclei (FIG. 5E) from those of the whole cell (FIG. 5D). The raw spectra from each segment were quality tested using a Python routine adapted from the Bruker OPUS software. The test controlled for signal to noise ratio (SNR) and signal to water ratio (SWR) to allow selection of spectra that fit the robust criteria to be included in the spectral biomarker (FIG. 5G). The spectra were subsequently pre-processed to reduce other artifacts that occurred during the acquisition (FIG. 5H), as described in the methods section. Corrected spectra are displayed as second derivative curves throughout the results.

FIGS. 5A-5K. Segmentation reveals differences in the lipid features in the WT and HD astrocytes FTIR signatures. Local Ostu's filter was applied to determine the background from the entire cell (FIG. 5A) or nucleus (FIG. 5B, shown in magenta). Seed points were used to localize cells from their estimated center (FIG. 5C, red dots). Seed watershed segmentation was applied to whole cells (FIG. 5D) and nuclei (FIG. 5E). Seed watershed segmentation was applied to the cytoplasm of the cells (FIG. 5F, entire cell pixels minus nucleus pixels). Scale bars=100 μm. An example of raw extracted whole astrocyte mean spectra before (left of FIG. 5G) and after (right of FIG. 5G) quality testing (QT) and pre-processing (FIG. 5H). Whole cell (FIG. 5I), nucleus (FIG. 5J), and cytoplasm (FIG. 5K) average spectra of WT and HD SV40T CBL astrocytes. For visual purpose 2^{nd} derivative normalized spectra are displayed between 3050-2800 cm⁻¹ (lipid-rich region) and 1800-900 cm⁻¹ (“fingerprint” region).

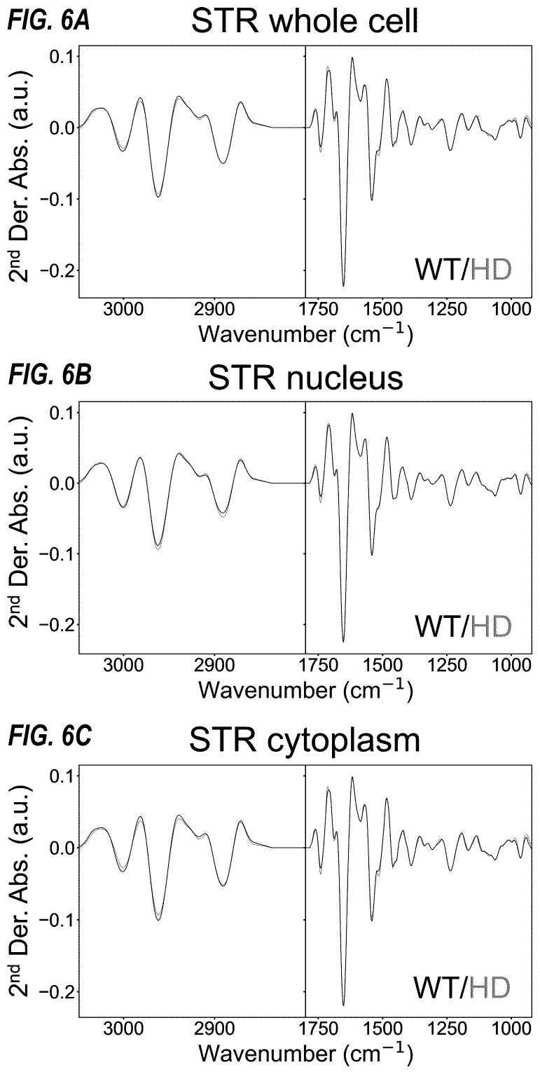

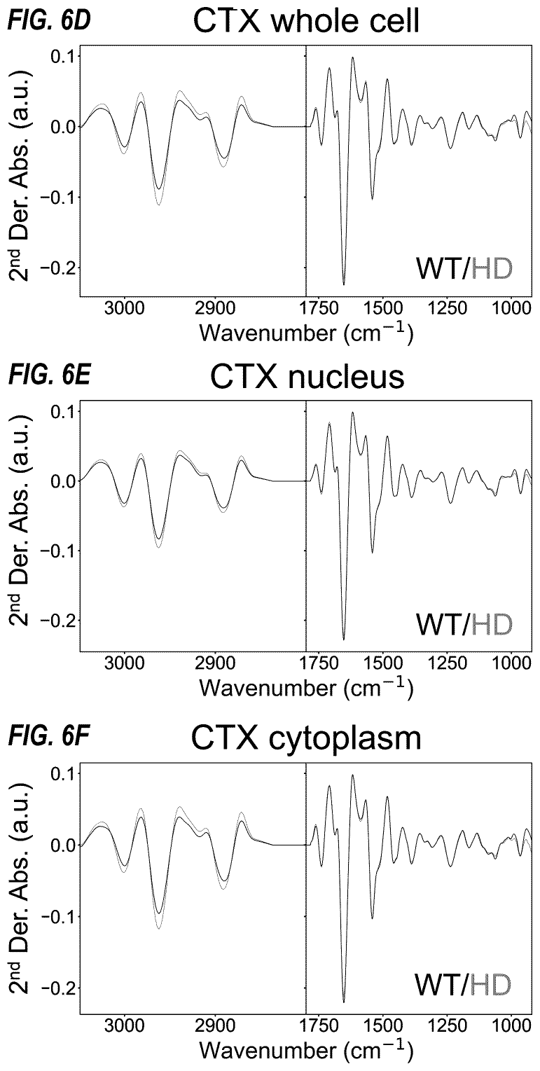

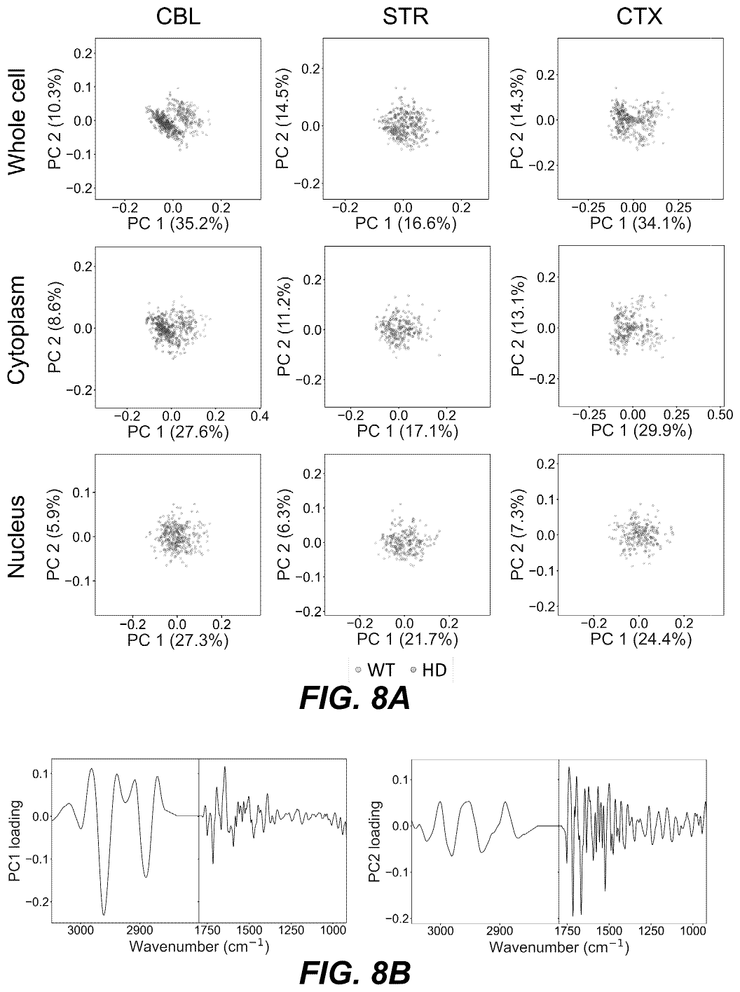

Indeed, in all cell segments, the mean absorbance spectra plotted as a second derivative, were different for WT and HD astrocytes (FIGS. 5I-5K, FIGS. 6A-6F), particularly in the lipid portion of the spectrum. For all three brain regions (CBL, STR and CTX), these differences are shown in the magnified views of spectra in then the 3050-2800 cm⁻¹ region, originating from mainly lipids and the “fingerprint” (1800-900 cm⁻¹) region (FIGS. 5I-5K, FIGS. 6A-6F). The “fingerprint” region comprises spectral features from lipids, but also contains features for proteins (amide bands), nucleic acids and carbohydrates (FIG. 1A). Whether cell segmentation mattered in the disease prediction was tested. Each sample was classified by clustering the mean spectrum from each cell segment (either nucleus, cytoplasm, or whole cell). Class assignment was evaluated by clustering using either unsupervised Principal Component Analysis (PCA) (FIG. 8A), or unsupervised non-linear Uniform Manifold Approximation and Projection (UMAP) method (FIGS. 7A-7I). While PCA assigns equal weights to all pairwise linear distances, UMAP is a non-linear method. Plots are unitless and reflect closest datapoints to define the clusters (FIGS. 7A-7I). Using either of these clustering techniques, biological classes were determined by the distance between the cluster centers. If samples are of distinct classes, the clusters would have little to no overlap. Indeed, the FTIR signature's ability to distinguish control and disease states critically depended on the choice of the cell segment. For P2 astrocytes, the clustered spectra from disease or control astrocytes were well separated and predicted disease class in the three brain regions tested if the features were extracted from whole cells (FIGS. 7A-7C) or from cytoplasm segments (FIGS. 7D-7F), both of which contain the lipid-rich plasma membrane. In contrast, clusters from nuclear segments significantly overlapped and consistently worsened the prediction (FIGS. 7G-7I). This was the case in both the UMAP (FIGS. 7A-7J) and PCA (FIGS. 8A-8B) plots. PC loadings (FIG. 8A) confirmed that sample (whole cells or cytoplasm segment) discrimination was based on lipid features (3050-2800 cm⁻¹) and on spectral features in the “fingerprint region” lipid peaks (1740 cm⁻¹, 1455 cm⁻¹) and protein features at 1655 and 1535 cm⁻¹ (amide I/II bands). Although changes to lipids are not unique to HD, their contribution to the disease signature in P2 astrocytes was significant. These molecules are not only vital to the health of the central nervous system, but lipids also are disrupted in Huntington's disease.

FIGS. 6A-6F. Segmented cell spectra of striatum and cerebellum astrocytes. Whole cell, nucleus, and cytoplasm average spectra of WT and HD SV40T STR (FIGS. 6A-6C) and CTX (FIGS. 6D-6F) astrocytes. For visual purpose 2^{nd} derivative normalized spectra are displayed between 3050-2800 cm⁻¹ (lipid-rich region) and 1800-900 cm⁻¹ (“fingerprint” region).

FIGS. 7A-7J. Spectral phenotyping accurately predicts (or determines) disease class in HD astrocytes. UMAP clustering and classification derived from segmented whole cell (FIGS. 7A-7C), cytoplasm (FIGS. 7D-7F) or nucleus (FIGS. 7G-7I) for three regions of the brain CBL (FIGS. 7A, 7D, and 7G), STR (FIGS. 7B, 7E, and 7H) and CTX (FIGS. 7C, 7F, and 7I). FIG. 7J. Confusion matrices corresponding to each UMAP shown in FIGS. 7A-7I. The predicted and actual classification results for HD and WT astrocytes in the whole cell, cytoplasm, and nucleus for all three brain regions are listed in Table 1.

FIGS. 8A-8B. PCA clustering distinguishes HD from WT for the three brain regions as in FIGS. 7A-7J. FIG. 8A. PCA plots corresponding to the UMAP analysis for the three brain regions performed in FIGS. 4A-4F. FIG. 8B. PC1 (left) and PC2 (right) loading for the WT and HD samples from the CBL whole cell PCA (top left corner). PC loadings showed that lipid features (PC1 loading) and amide bands (PC2 loading) had a high contribution to the WT and HD cell discrimination.

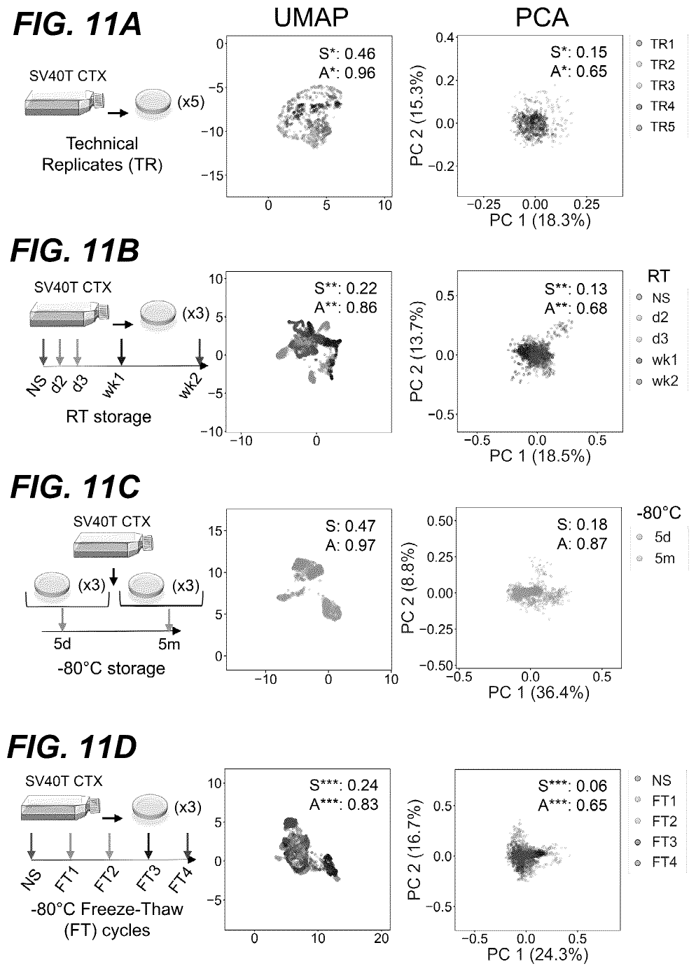

The quality of the classification was quantified in the PCA/UMAP analysis by a Silhouette score (S), which is a metric for how close each point in one cluster (cohesion) is to its neighboring clusters (separation) (Table 1). The metric is calculated on a −1.0 to 1.0 scale with a higher score indicating datapoints that are closer to their own clusters than to other clusters. Indeed, the S for disease prediction (whole cell or cytoplasm) from all three brain regions ranged from 0.4 to greater than 0.7, indicating a good distinction between the two classes (Table 1). In contrast, the S for the nuclear segment ranged from 0.09 to 0.22 indicating that the control and disease signatures were not well-resolved. The spectral distinctions from the second derivative absorbance curves in all three regions are shown (FIGS. 7A-7J). Shuffling and permutation of the FTIR datasets in each region confirmed that the classification was robust (p<0.001) for cytoplasm and whole cell analysis (Table 1). UMAP, by its distance emphasis, was sensitive enough to reveal small differences among technical and biological replicates, which were not necessarily identified using PCA (FIG. 11A). Nonetheless, using either approach, the disease prediction was robust (FIGS. 7A-7J, Table 1, FIGS. 8A-8B, Table 2) and reproducible in technical and biological preparations used throughout the analysis.

TABLE 1
Metrics for spectral classification (from FIGS. 7A-7F).

| | WT vs HD CBL | | | WT vs HD STR | | | WT vs HD CTX | | |
|---|---|---|---|---|---|---|---|---|---|
| | NUC | CYT | CELL | NUC | CYT | CELL | NUC | CYT | CELL |
| S^{a} | 0.09 | 0.62* | 0.78* | 0.11* | 0.39* | 0.52* | 0.22* | 0.35* | 0.41* |
| Sens^{b} | 0.87 | 1.00 | 1.00 | 0.88 | 0.93 | 0.96 | 0.87 | 0.95 | 0.99 |
| Sens^{c} | 0.42 | 0.99 | 1.00 | 0.70 | 0.97 | 0.99 | 0.75 | 0.91 | 0.93 |
| A^{d} | 0.72 | 1.00 | 1.00 | 0.81 | 0.95 | 0.98 | 0.81 | 0.93 | 0.95 |

*p-value: <0.001.
^{a}S, silhouette score;
^{b}Sens, sensitivity;
^{c}Spec, specificity;
^{d}A, accuracy.

TABLE 2
Metrics for spectral classification (from FIGS. 7A-7F; FIGS. 8A-8B).

| | WT vs HD CBL | | | WT vs HD STR | | | WT vs HD CTX | | |
|---|---|---|---|---|---|---|---|---|---|
| | CYT | CELL | NUC | CYT | CELL | NUC | CYT | CELL | CYT |
| S^{a} | 0.45* | 0.48* | 0.13* | 0.26* | 0.33* | 0.19* | 0.37* | 0.41* | 0.45* |
| Sens^{b} | 0.99 | 0.99 | 0.80 | 0.86 | 0.93 | 0.86 | 0.96 | 0.98 | 0.99 |
| Spec^{c} | 0.93 | 0.98 | 0.67 | 0.88 | 0.97 | 0.70 | 0.88 | 0.92 | 0.93 |
| A^{d} | 0.97 | 0.99 | 0.75 | 0.88 | 0.95 | 0.78 | 0.92 | 0.95 | 0.97 |

*p-value: <0.001.
^{a}S, silhouette score;
^{b}Sens, sensitivity;
^{c}Spec, specificity;
^{d}A, accuracy.

### Spectral Phenotyping is Accurate.

The quality and accuracy of the classification was established from a confusion matrix (FIG. 7J) using a k-nearest neighbor (k_{NN}) statistical model. The confusion matrix is a signature classifier, which considers all data instances as either positive (disease) or negative (controls). The results of the confusion matrix for all three regions are shown and key statistical metrics are summarized (FIG. 7J). Indeed, the number of false positive and false negative assignments was consistently low, and accuracy (A) of correct assignment was over 90% for most samples using cytoplasmic or whole cell segments. The high sensitivity and specificity also indicated that a high proportion of disease or control samples were classified as such (Table 1). Thus, the disease prediction from unsupervised PCA (Table 2) and UMAP was accurate.

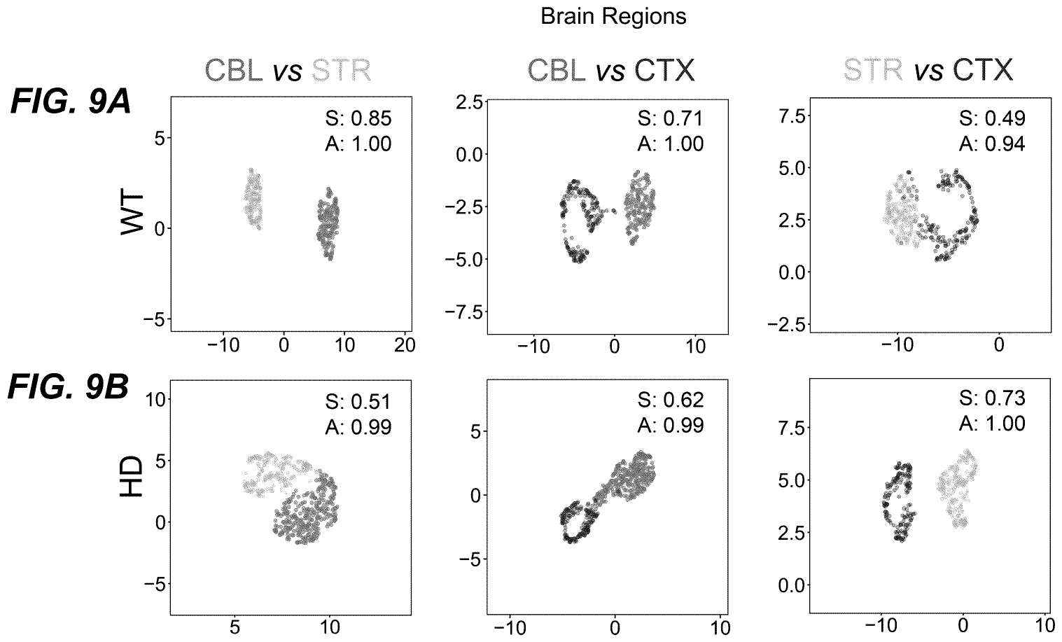

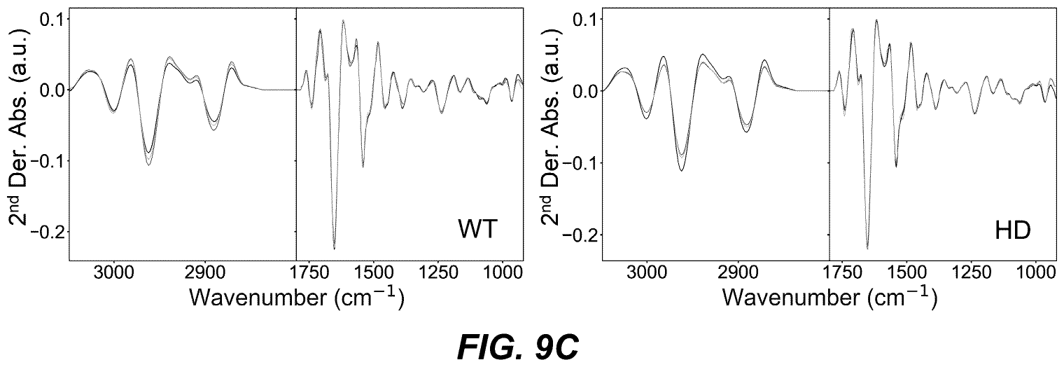

Whether the FTIR signature was sensitive enough to discriminate among astrocytes from distinct brain regions from either WT or HD animals was evaluated (FIGS. 9A-9C). This was a more stringent test of classification since the cells to be evaluated were of the same type (astrocytes) and shared the same genotype. The FTIR signature would differ only if the features reflected the spatial origins of the astrocytes. Surprisingly, the P2 astrocytes from WT mice as well as their HD littermates were characterized by a spatial identity as early as two days after birth (FIGS. 9A and 9B). Thus, FTIR signatures recognized subtle differences (FIG. 9C) in the modifications among cellular molecules that defined their regional position. The FTIR signature predicted disease class in astrocytes at very early ages, consistent with growing evidence that HD is a developmental disorder. The cluster separation among regions was good to excellent, with S ranging from around 0.4 to 0.85 depending on the regional comparison (FIGS. 9A and 9B). Collectively, the results provided evidence that spectral phenotyping was able to predict disease class of astrocytes with high probability using a unique FTIR signature as the biomarker. Not only did FTIR signatures accurately predicted disease class, but the FTIR signatures were able to discriminate between control and disease astrocytes, which were isolated as early as 2 days after birth and displayed no obvious phenotypic differences.

FIGS. 9A-9C. Astrocytes have regional signatures that are distinguishable by their FTIR signatures. FIGS. 9A-9B. Pairwise classification of astrocytes isolated from the CBL, STR and CTX brain regions of SV40T WT (FIG. 9A) or HD (FIG. 9B) animals by UMAPs of 2^{nd} derivative normalized absorbance FTIR spectra (whole cells). FIG. 9C. Average 2^{nd} derivative normalized spectra of WT (left) and HD (right) SV40T astrocytes from the CBL (blue), STR (orange), CTX (green) brain regions. Spectra are displayed between 3050-2800 cm⁻¹ (lipid-rich region) and 1800-900 cm⁻¹ (“fingerprint” region). S, silhouette score (p-value: <0.001); A, accuracy.

### The Disease Signatures are Reproducible.

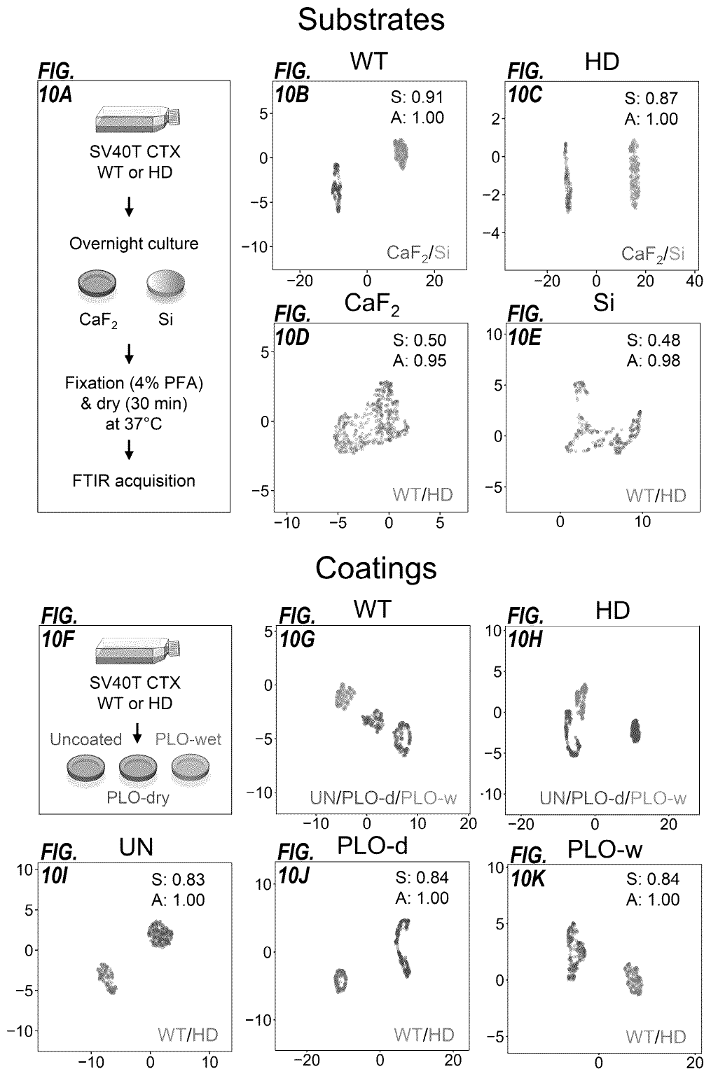

The astrocytes samples were isolated from distinct litters of pups and the slides were stored between measurements. To ensure that the FTIR classification was robust, the reproducibility of the FTIR signature for cell preparations under relevant condition of temperature, storage, and slide preparation was measured. The impact of slide substrate type (FIGS. 10A-10E), slide coating (FIGS. 10F-10K), sample storage time and storage temperature (FIGS. 11A-11D) on the accuracy of the FTIR disease prediction were tested. FTIR spectra were acquired using transmission mode, which requires IR light to pass through the slide and sample. Calcium fluoride (CaF₂) or silicon (Si) are typical substrates for this purpose (FIG. 10A). In the experiments, CaF₂ was used most often. Although the choice of substrate had an impact on the resulting FTIR signature (FIGS. 10B and 10C), WT and HD discrimination was successful using spectral phenotyping as long as samples were measured and compared using the same substrate (FIGS. 10D and 10E). The predictions had a good S and high A (FIGS. 10D and 10E). Slide coatings are not always needed but are often used to improve cell adherence to the substrate. A common coating is poly-L-ornithine (PLO), which is used wet (PLO-w) or dry (PLO-d) in various preparation protocols. Samples were prepared as in FIG. 10A, and whether cells layered onto wet or dry PLO coating altered the disease prediction relative to uncoated slides was tested (FIG. 10F). Although the slide coatings themselves had an impact on the resulting FTIR signature (FIGS. 10G and 10H), WT and HD discrimination was successful independent of coating, as long as the compared samples were measured under the same conditions (FIGS. 10I-10K).

FIGS. 10A-10K. FTIR substrates and coatings have an influence on cell spectra without altering disease/control classification. FIG. 10A. Experimental protocol schematic representing SV40T CTX WT or HD astrocytes cultured overnight on CaF₂ and Si substrates. Cells were fixed and dried prior to the FTIR acquisition. FIGS. 10B-10C. UMAP clustering results of WT (FIG. 10B) or HD (FIG. 10C) cells grown on CaF₂ and Si substrates. FIGS. 10D-10E. UMAP classification of WT and HD astrocytes grown on either CaF₂ (FIG. 10D) or Si (FIG. 10E) substrates. FIG. 10F. Schematic of substrate coating effect experiment following the same procedure as in FIG. 10. SV40T CTX WT or HD astrocytes were cultured overnight onto CaF₂ substrates uncoated (UN), with poly-L-ornithine dry (PLO-d) or poly-L-ornithine wet (PLO-w) coatings. FIGS. 10G-10H. UMAP clustering results for all three coatings on CaF₂ substrates for WT (FIG. 10G) or HD (FIG. 10H) cells. FIGS. 10I-10K. UMAP classification of WT and HD astrocytes grown on CaF₂ substrates uncoated (FIG. 10I) or coated with PLO-d (FIG. 10J) and PLO-w (FIG. 10K). All UMAP analyses were performed on 2^{nd} derivative normalized absorbance FTIR spectra of whole cells. S, silhouette score (p-value: <0.001); A, accuracy.

The impact of sample storage on the robustness of the disease prediction was determined. Slides were prepared and stored at RT (FIG. 11B) or −80° C. (FIG. 11C), for various periods from which the FTIR signature was measured before and after storage. Sample storage at room temperature (RT) yielded a relatively low S indicating significant overlap after a day of storage up to two weeks (FIG. 11B). The spectral signatures were not reproducible during long term storage (5 months) at −80° C. (i.e., class separation) (FIG. 11C). However, signatures were stable for at least two weeks if samples measured at RT were returned to storage at −80° C. between subsequent measurements (freeze-thaw) (FIG. 11D). Although there are inevitable chemical changes that occur when cells are fixed, as previously reported, fixation did not impair disease classification as long as both samples were fixed under the same conditions. Collectively, these results defined conditions for sample preparation that resulted in robust measurements, such as the FTIR samples being layered onto uncoated calcium fluoride slides, dried, fixed and stored at RT during the experiment.

FIGS. 11A-111D. Best practice conditions for reproducibility of the FTIR signatures measured under various conditions. Reproducibility of cell spectra under various conditions was assessed by UMAP (left) and PCA (right) analysis. FIG. 11A. Technical replicates (TR) reproducibility. The S* and A* values were calculated for TR1 and TR5. FIG. 11B. Storage at RT. The S** and A** values are calculated for NS (no storage) and wk2. FIG. 11C. Storage at −80° C.; the S and A values are calculated for 5 days (d) and 5 months (m). FIG. 11D. Samples not stored (NS) compared to measurements after Freeze (−80° C.) and thaw (RT) cycles. The S*** and A*** values calculated for NS and FT4.

### FTIR Phenotyping is a General Use Tool for Disease Prediction in Human Cells.

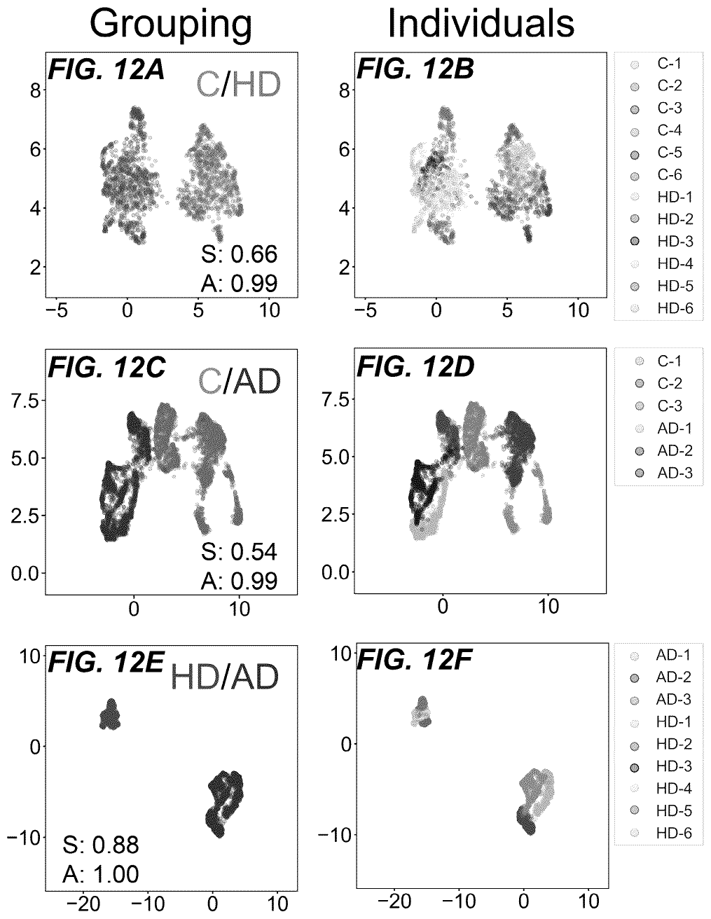

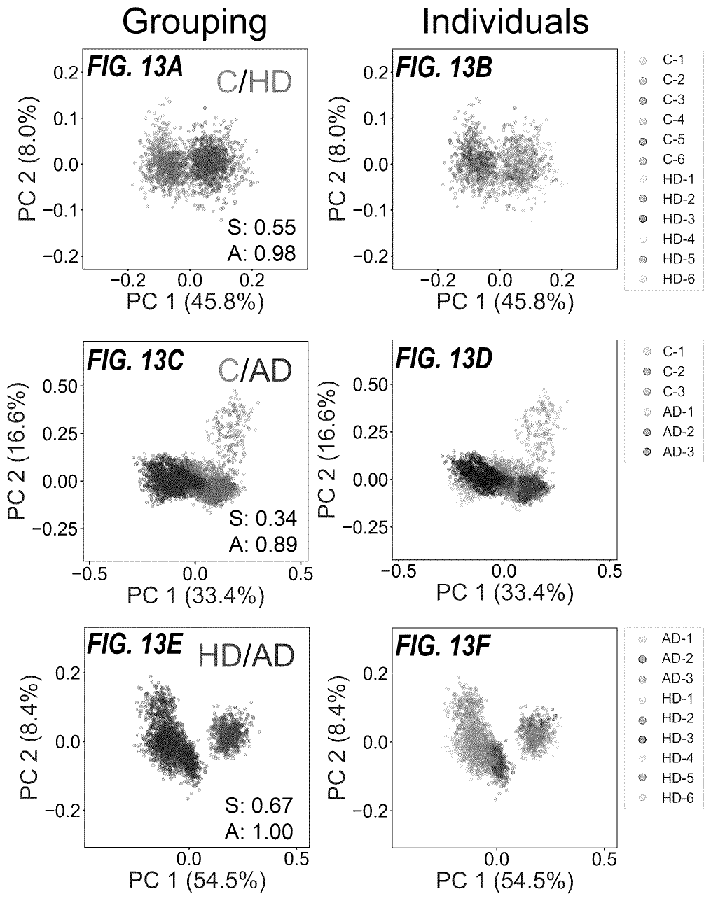

In practice, the usefulness of FTIR spectral phenotyping as a biomarker is its ability to accurately classify human disease cells. Since the brain is not accessible for analysis, whether HD patient fibroblasts might be used as surrogates was considered. The premise being that these cells shared the same genotype with HD brain cells and might undergo chemical changes that tracked with disease. HD human fibroblast samples were obtained from the Coriell repository. The demographics of each patient are listed (Table 3). Spectral phenotyping was evaluated as a classifier by evaluating either pooled samples (FIG. 12A) or as individual samples (FIG. 12B). PCA (FIGS. 13A-13F) or UMAP (FIGS. 12A-12F) clustering was used to determine the disease class.

FIGS. 12A-12F. Spectral phenotyping can predict human neurodegenerative disease class from fibroblasts. FTIR spectra from human skin fibroblasts of controls (C) versus Huntington's disease (HD) (FIGS. 12A and 12B), controls (C) versus Alzheimer's disease (AD) (FIGS. 12C and 12D) or a comparison of HD and AD (FIGS. 12E and 12F) were evaluated by UMAP. The UMAP plots are the results of either pooled control or pooled disease samples (FIGS. 12A, 12C, and 12E), or displayed per individuals (FIGS. 12B, 12D, and 12F). All UMAP analyses were performed on 2^{nd} derivative normalized FTIR spectra of whole cells. S, silhouette score (p-value: <0.001); A, accuracy.

FIGS. 13A-13F. The PCA analysis corresponding to the UMAP analysis (FIGS. 12A-12F) for control and various disease fibroblast samples. FTIR spectra from human skin fibroblasts of controls (C) and Huntington's disease (HD) (FIGS. 13A and 13B), controls (C) and Alzheimer's disease (AD) (FIGS. 13C and 13D), and HD versus AD (FIGS. 13E and 13F) patients were evaluated by PCA. The PCA plots are the results of either pooled control or pooled disease samples (FIGS. 13A, 13C, and 13E), or displayed per individuals (FIGS. 13B, 13D, and 13). All PCA analyses were performed on 2^{nd} derivative normalized FTIR spectra of whole cells. S: silhouette score (p-value: <0.001), A: accuracy.

TABLE 3
Demographics for disease patients and controls.

| | | | | Age^{a} | | | |
|---|---|---|---|---|---|---|---|
| Label | Sample ID | Disease | Sex | (yr) | Ethnicity | Cell origin | Brief description |
| HD-1 | GM05030 | HD | Male | 56 | Caucasian | NS | Choreic movements, |
| | | | | | | | dementia. |
| HD-2 | GM05031 | HD | Male | 60 | Caucasian | NS | Choreic movements, |
| | | | | | | | dementia. |
| HD-3 | GM04476 | HD | Male | 57 | Caucasian | NS | Onset at age 45, |
| | | | | | | | difficulty in ambulation |
| | | | | | | | with frequent falls. |
| HD-4 | GM04691 | HD | Male | 31 | Caucasian | NS | Onset at age 22, |
| | | | | | | | prominent laughing, |
| | | | | | | | involuntary vocalizations, |
| | | | | | | | dementia, dystonia. |
| HD-5 | GM04777 | HD | Male | 53 | Caucasian | NS | Clinically affected. |
| HD-6* | GM04693 | HD | Male | 33 | Caucasian | NS | Onset at age 41. |
| AD-1 | AG07377 | AD | Male | 60 | Caucasian | Skin (Arm) | Progressive mental |
| | | | | | | | deterioration since age |
| | | | | | | | 51, development of |
| | | | | | | | aggressive behaviour. |
| | | | | | | | Moderate to marked |
| | | | | | | | cortical atrophy (CT |
| | | | | | | | scan). NFH. |
| AD-2 | AG06262 | AD | Male | 66 | Caucasian | Skin (Arm) | Progressive dementia |
| | | | | | | | with memory deficits, |
| | | | | | | | requiring hospitalization |
| | | | | | | | at age 62. Nonfocal |
| | | | | | | | cortical atrophy (CT |
| | | | | | | | scan, age 59). NFH. |
| AD-3 | AG07376 | AD | Male | 60 | Caucasian | Skin (Arm) | Progressive intellectual |
| | | | | | | | deterioration since age |
| | | | | | | | 54. Severe cerebral |
| | | | | | | | atrophy (CT scan). NFH. |
| C-1 | AG08125 | Control | Male | 64 | Caucasian | Skin (Arm) | Unaffected. |
| C-2 | AG07623 | Control | Male | 60 | Caucasian | Skin (Arm) | Unaffected. |
| C-3 | AG08543 | Control | Male | 62 | Caucasian | Skin (Arm) | Unaffected. |
| C-4 | GM00288 | Control | Male | 64 | Caucasian | Skin (Arm) | Unaffected. |
| C-5 | GM09918 | Control | Male | 78 | Caucasian | Skin (Arm) | Unaffected. |
| C-6 | GM03658 | Control | Male | 68 | Caucasian | Skin (Arm) | Unaffected. |

All cell lines were obtained from the Coriell repository.
^{a}At sampling;
NS: Not stated;
NFH: no family history.
*collected before the onset of symptoms.

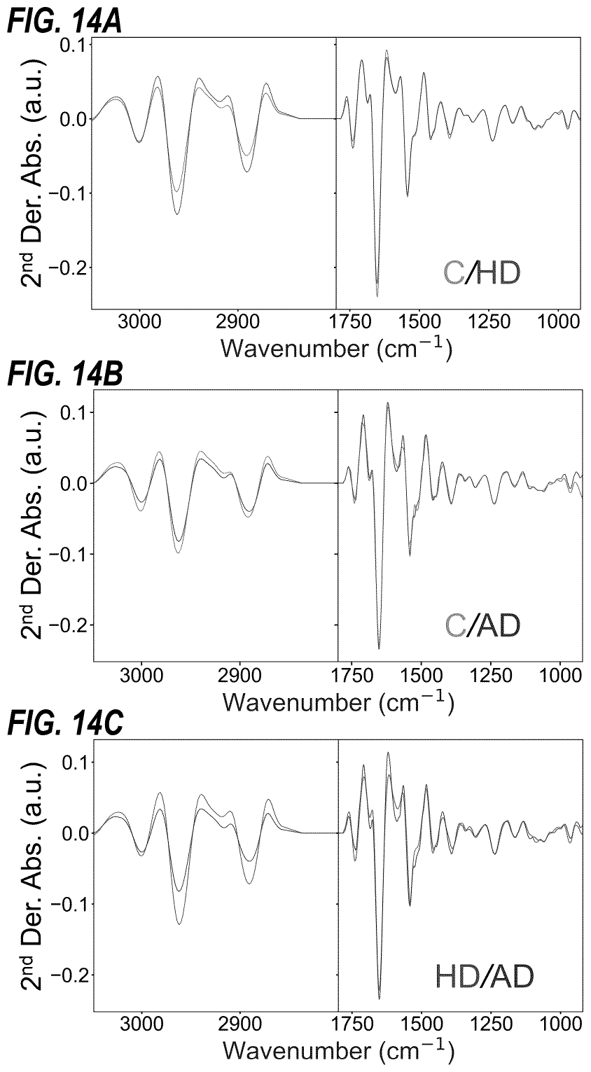

All samples were gender matched (male). For HD, most of the control and patients were of similar age (around 60 years), but two HD patients were younger (around 35 years) than controls and one control was older (78 years) than the HD patients. Despite the age variations, the disease classification, as judged by either UMAP (FIG. 12A) or PCA (FIG. 13A), was robust for human HD fibroblasts, with an S of 0.66 and high A of 0.99 (FIG. 12A). Mean spectra for control and HD fibroblasts are displayed (FIGS. 14A-14C). The results suggested that there were at least some chemical features that are shared among HD patients, which were distinct from those of controls. Although individual HD patients and controls often formed their own clusters (FIG. 12B), these samples grouped within larger clusters according to disease class (FIGS. 12A and 12B). Thus, the human HD fibroblast results added significance to spectral phenotyping since spectral phenotyping was effective in classifying HD class across species, including mouse (FIGS. 7A-7F) or human (FIG. 12A) harboring the mutant disease gene. Although more variation among human samples relative to those previously measured in the mouse samples was expected (FIGS. 7A-7F), the chemical biomarker for HD cells distinguished disease class regardless of species or cell type. The predictions for human fibroblasts (FIG. 12A) and mouse astrocytes (FIGS. 7A-7F) were equally robust.

FIGS. 14A-14C. HD and AD spectral signatures. Mean second derivative normalized FTIR spectra (whole cells) of HD (FIG. 14A) and AD (FIG. 14B) from FIGS. 12A-12F and FIGS. 13A-13F, compared to the signature of control (C) cells. FIG. 14C. Direct comparison of the HD and AD spectral signatures. For visual purpose 2^{nd} derivative normalized spectra are displayed between 3050-2800 cm⁻¹ (lipid-rich region) and 1800-900 cm⁻¹ (“fingerprint” region).

The accuracy of disease classification using the FTIR biomarker was not limited to HD. Three AD human samples were also classified relative to age and gender matched controls. All male AD patients were between 60 and 66 years as compared to the male controls which ranged from 60-78 years. Like the HD results, all three AD patient samples clustered as a group that was distinct from controls even though the underlying mutations were unknown for any sample (FIG. 12C). As with HD, individual control and AD patients were resolvable from each other (FIG. 12D) as judged by either PCA (FIG. 13D) or UMAP (FIG. 12D), but overall, the samples grouped according to their disease class, validating the disease prediction usefulness of fibroblasts. HD and AD are late onset diseases but differ significantly in that the first is due to a dominant and fatal genetic disorder, while in the latter the underlying mutation is unknown for most patients and death does not always occur from the disease. Yet, robust classification of human fibroblasts from each of these neurodegenerative diseases was possible even in what visually appeared to be homogeneous and indistinguishable cultures. Thus, the unique FTIR chemical biomarker was accurate in predicting disease class in cells of different species, of distinct types, and between two neurodegenerative diseases.

### Methods

Animals and cell lines. Breeding and use of HhdQ(150/150) and C57Bl6J mice was performed as reported previously. All procedures involving animals were approved by the Lawrence Berkeley National Laboratory Animal Welfare and Research Committee and performed in accordance with the relevant guidelines and regulations. The use of live animal was carried out in compliance with the ARRIVE guidelines. Established human cell lines used in this study include AD and HD human fibroblasts obtained from the Coriell repository. The demographics and phenotypic data are reported for each cell line in Table 3.

Dissections and isolation of primary astrocyte cultures. Mouse primary astrocytes were isolated from various brain regions as the follows. Intact brains were collected from postnatal day 1-3 pups (called P2) for either genotype (HhdQ(150/150) or C57Bl6J mice). Brain regions (cerebellum, striatum and cortex) were isolated in a solution of Phosphate Buffer Saline (PBS) on ice. The regions of 4-7 pups of each genotype were pooled and digested in 10 mL 0.25% Trypsin-Ethylenediaminetetraacetic acid (EDTA) (Gibco 25300056) in PBS for 15 min at 37° C. Tissue pieces were pelleted (5 min, 300 rcf, room temperature (RT)) and then gently triturated 20-30 times in pre-warmed potent media (DMEM (Gibco 10569044), 20% FBS (JRS 43635), 2.5 mM glucose, 2 mM sodium pyruvate, 2 mM glutamax, 1× non-essential amino acids (Quality Biologicals 116-078-721EA), and 1× antibiotic/antimycotic (Gibco #15240062) using a 5 mL pipet, to dissociate into single cells. Each cell suspension was plated into poly-L-ornithine (VWR 103701-204) coated T75 culture flasks and cultured for 7-10 days (at 37° C., 5% CO₂), with media exchanges every 2-3 days. Cells were re-passaged twice to enrich for astrocytes. Astrocyte cell purity and homogeneity was tested by immunofluorescent analysis using anti-Glial Fibrillary Acidic Protein (GLAST) antibody (Invitrogen SPM498).

SV40T immortalized astrocyte cultures. Primary cells were transformed with SV40 Large T antigen (ABM LV660), according to the manufacturer's protocol, to create clonally derived immortalized cell lines. Briefly, logarithmically growing primary astrocytes in 6 well dishes with 1 mL potent media, were treated with 1×10⁶ units of high-titer SV40T lentiviral stock (ABM LV660), 5 μg/mL polybrene (EMD Millipore TR-1003-G) and 20 μL of ViralPlus Transduction Enhancer (ABM G698). Following 1 day of culture, cells were washed with fresh media and allowed to grow for an additional 3 days. Cells were then replated into two 10 cm diameter dishes and cultured for 4-6 days with 0.1 μg/mL puromycin. Individual clones were selected using cloning discs (Sigma Z374431) and grown up individually.

Immunocytochemistry. Cells were fixed in freshly prepared 4% paraformaldehyde (PFA) (10 min at RT in the dark), then incubated with 100 mM Glycine, 0.1% Triton X-100, 0.05% Tween-20 in PBS (5 min), and blocked (1-2 h) in blocking solution (PBS, 3% Bovine Serum Albumin (BSA), 3% goat serum, 3% donkey serum, 0.03% triton X-100). Primary antibody (1:500 rabbit anti-GLAST (Invitrogen SPM498)) diluted in 10% blocking solution/PBS was added for 1 h, followed by 3 washes with PBS (5 min each). Appropriate secondary antibody (1:1,000 donkey anti-rabbit Alexa 546 (Invitrogen A10040)), diluted in 10% blocking solution/PBS was then applied along with 0.5 μM DAPI (30 min) for nuclear staining followed by 2 washes in PBS. Slides were coated with Aqua Polymount (Fisher Scientific NC9439247), covered by a #1.5 coverslip, sealed with clear nailpolish and stored (−20° C.). Slides were imaged using a Zeiss 710 confocal microscope at 1 A.U., using either 20× (0.8N/A)/air or 63× (1.4N/A)/oil lenses.

Grip test. For the grip strength-endurance test, mice were lowered onto a parallel rod (diameter <0.25 cm) placed 50 cm above a padded surface. The mice were allowed to grab the rod with their forelimbs, after which they were released and scored for length of time they could hold onto the bar (maximum 30 sec). Mice were tested consecutively 3 times at each age. The maximum length of time they were able to hold on was recorded for analysis.

MitoTracker Cell Staining. Staining was done according to the manufacturer's instructions. Briefly, astrocyte cells were plated and allowed to grow in growth media until they reached 60-70% confluence. Media was removed and replaced with fresh media containing 100 nM Mitotracker Green FM. Cells were incubated for 30 min at 37° C. and 5% CO₂ after which the media was removed, cells were washed with PBS and later fixed with 4% PFA containing 300 nM DAPI for 15 min. Cells were then re-washed with PBS and imaged.

Western blot Analysis. Astrocytes were plated on 10 cm cell culture plates in Growth Medium, transfected, and allowed to express heterologous proteins (16-20 hrs). Cells were then gently washed with ice cold PBS (pH 7.4) and scraped off in Lysis Buffer (200 ul RIPA buffer (ThermoFisher #89900) supplemented with HALT Protease Inhibitor (ThermoFisher #7842) and 5 μg/mL DNase I (ThermoFisher #18047-019)), triturated (20× with 200 μL pipet) and sonicated (3×15 sec on ice). Protein concentration was determined using Pierce 660 nm Protein Assay Kit (ThemoFisher #22662) and relevant protein amounts (5-15 μg) were brought up in NuPage LDS Sample Buffer (ThermoFisher #NP0007) and NuPage Sample Reducing Agent (ThermoFisher #NP0004). Samples were heated at 95° C. for 10 min and debris was pelleted (20,000 rcf, 10 min, room temperature (r.t.)). Samples were resolved on either 4-12%, 8-16% or 4-10% Novex Tris-Glycine SDS-Page mini gels (ThermoFisher) in Novex Tris-Glycine SDS Running Buffer at r.t. and transferred onto nitrocellulose membranes (0.2 m) using BioRad Trans-blot Turbo Transfer System (according to manufacturer's protocol). Blots were washed with PBST (pH 7.4), general protein visualized using Ponceau S (SigmaAldrich #P7170), then rewashed with PBST. Blots were blocked in Blocking Buffer (5% Non-Fat Dry Milk (NFDM) in PBST (pH 7.4)) then probed with primary antibody (1:10,000 in Blocking Buffer) in a sealed pouch, with rocking for 1 hr at RT. Blots were washed (3×) 10 min using PBST with rocking, and probed with secondary HRP labelled antibody (1:15,000 in Blocking) in a sealed pouch, with rocking for 30 min at RT prior to final washes (3×) 10 min using PBST with rocking. HRP was visualized using either the ECL Prime or ECL Select Chemiluminescent Detection Kits (SigmaAldrich) (according to manufacturer's protocols) and imaged on a BioRad VersaDoc Imaging System. Primary Antibodies used were mouse anti-Htt (Millipore #MAB-2166)(htt), Mouse anti-polyQ (DSHB #MW1)(mhtt), anti-GAPDH Goat anti-GAPDH (Genscript #A00191). The secondary antibodies were Goat anti-Mouse HRP conjugate (Thermo Fisher Sci #G21040) and Rabbit anti-Goat HRP conjugate (Thermo Fisher Sci #31402)

Sample preparation for spectral analysis. Dissociated cells in potent media were plated onto IR sterile substrates (25 mm×1 mm calcium fluoride (CaF₂) or silicon (Si) windows (Crystran Ltd, UK) inside wells of a 6 well plate. Substrates were either uncoated or coated with poly-L-ornithine (VWR 103701-204). ‘Wet’ coating involved incubating the substrates with 0.01% poly-L-ornithine for 30 min at room temperature (RT) and washing twice with PBS. ‘Dry’ coating involved incubation with poly-L-ornithine, removal of the solution by pipet and allowing the substrate to dry inside a laminar flow hood. Cells were grown 1-2 days (at 37° C., 5% CO₂). The media was removed, and slides were rinsed twice with PBS before cell fixation with 4% PFA in PBS for 10 min. Following fixation, the slides were rinsed with ultra-pure water (MilliQ water). The washed cells were dried at 37° C. for 30 min and kept in dark boxes with desiccants at either RT or in an −80° C. freezer prior to multispectral analysis.

### The Methodology for Spectral Phenotyping.

(a) FTIR spectral imaging acquisitions. FTIR spectral images were collected using an Agilent Cary 670 FTIR spectrometer coupled to an Agilent Cary 620 FTIR microscope (Agilent Technologies, USA) with a 128 by 128 pixel liquid nitrogen cooled Mercury Cadmium Telluride (MCT) Focal Plane Array (FPA) detector. The Agilent system was also equipped with an in-built purging system allowing the maintenance of a low relative humidity during acquisitions. Images were obtained from multiple tiles of 704 m by 704 μm acquired with a 15× magnification objective and condenser resulting in a projected pixel size of 5.5 μm². Spectral data were collected using the Agilent Resolutions Pro software in the transmission mode, by the co-addition of 256 and 128 scans for the background and samples respectively, at a spectral resolution of 4 cm⁻¹ over the spectral range 4000-800 cm⁻¹.

(b) Segmentation. All spectral data were processed using a software program written in Python 3. The Otsu's threshold algorithm was used to delineate subcellular segments for the spectral analysis. Otsu's algorithm is a semi-automated thresholding approach to define foreground and background in a grayscale image. Since hyperspectral images were acquired, they were reduced into high contrast 2D images based on the integrated absorbance frequencies between 1670-1630 cm⁻¹ (amide I band) for each pixel (FIG. 1). For two classes, (e.g., foreground and background) the optimal threshold is chosen when Otsu's algorithm has maximized the inter-class variation. For all types of cells, this example used a modified Otsu's algorithm which allows for local thresholding of 2D images, by applying the same principle, but on user-defined (size and shape) disk shaped pixel blocks. This “dynamic thresholding” approach is useful when the background of the image is non-uniform. Then, individual cells and cell nuclei were defined using the seed-watershed algorithm for separating different objects in an image. The locations of nuclei centers were used as “seed points” in the watershed method, which is a topographic distance algorithm. From these seed points, “basins” are flooded and separated by “watershed” lines when they meet. These watershed lines correspond to the estimated edges of the basins. In this example case, this step was used to estimate the pixels of entire cells and cell nuclei. The cytoplasm pixels were derived by subtracting the designated nucleus pixels from those of the whole cell. Attributed nucleus and cytoplasm pixels were eroded by two pixels to enhance cytoplasm and nucleus or cell-cell delineation. Finally, a mean spectrum was computed from each cell segment.

(c) Quality testing. A quality test was applied to each spectrum using a routine adapted from the commercially available Bruker OPUS software. Extracted spectra were quality tested to control for absorbance (A), signal to noise ratio (SNR), and signal to water vapor ratio (SWR). In this example, the cutoff value for each parameter was calculated based on 3332 spectra (nuclei and cytoplasm) coming from 1666 fixed, cultured astrocytes. The lower and higher bound values for A were chosen arbitrarily to the mean absorbance±5 standard deviations. SNR was calculated from parameters S1 and S2 corresponding to the difference between the minimum and maximum value of the first derivative on the band 1600-1700 cm⁻¹ (amide I) and 960-1260 cm⁻¹ (sugar-ring), divided by the noise (N) intensity over the 2100-2000 cm⁻¹ region, where no absorbance is typically present in biological samples. Spectra were rejected when S1/N and S2/N were equal to the mean value of these equations±1 standard deviation. SWR was calculated from S1, S2 divided by the water vapor content (WVC) parameter which is the difference between the maximum and minimum value of the first derivative calculated between the 1847-1837 cm⁻¹ range, which exhibits a strong water vapor absorbance and no sample contribution. Spectra were rejected when S1/WVC and S2/WVC were equal to the mean value of these equations±1 standard deviation. Using these cutoff values, 80% of the 3332 spectra passed the quality test.

(d) Pre-processing. To extract the chemical information embedded in the absorbance values of the spectra, a technique was applied to minimize physical artifacts that might have occurred during the acquisition. Initially raw spectra which passed the quality test were cut and pre-processed over the 4000-900 cm⁻¹ range. Spectra were smoothed using the Savitzky-Golay method before applying a second derivative (21 points, 2^{nd} polynomial order) for baseline correction and spectral contrast optimization. Then, spectra were vector normalized to enable their comparison. To simplify spectral feature visualization, the pre-processed mean spectra were displayed between the lipid-rich (3050-2800 cm⁻¹) and the “fingerprint” (1800-900 cm⁻¹) regions.

(e) Biological classification and statistics. Biological classification was accomplished by clustering the data after dimensionality reduction using UMAP or PCA. The separation of the data into clusters indicates the different biological classes. PCA maximizes the linear (Euclidean distance) variance between spectra projected in 2D while UMAP is a topological method that optimizes the connectedness of spectra in the dataset. The quality of the clusters was defined by a Silhouette score (S), which is computed based on the mean intra-cluster distance (the distance between one cell and all others in the same cluster) and the mean distance between one cell and all other cells of the next nearest cluster (mean nearest-cluster distance). Individual spectra were classified using a k-nearest neighbor (k_{NN}) statistical model and accuracy was calculated from a confusion matrix (FIG. 7J, Table 1). In a k_{NN} model, the k training points that are nearest to each test datapoint are considered, and the predicted identity is the most commonly occurring label among those k points. The analysis was performed with k-fold cross validation with k=3. This means that each dataset was randomly shuffled and evenly split into 3 subsets. A k_{NN} model was trained on two of these subsets and evaluated on the third. As a final step, each of the subsets were merged and the datapoints were grouped according to control or disease and the number of correct and incorrect assignments was calculated. Thus, the confusion matrix summarizes the performance of the classifier, by considering all datapoints as either positive (disease) or negative (controls). A true positive (TP) is a sample which is correctly classified as HD (disease). A true negative (TN) refers to the samples without the mutant gene, which are correctly assigned as a WT (control). False positives (FP) are spectra from a control sample, which are incorrectly identified as a disease sample. A false negative (FN) is a disease sample, which is incorrectly classified as a control cell. Using these parameters, the accuracy (A) (Eq. 1), specificity (SP) (Eq. 2), and sensitivity (SEN) (Eq.3) were derived, respectively. Each parameter is scored from best (1.0) to worst (0).

$$A = ( T P + T N ) / ( T P + T N + F P + F N ) ; the number of correct assignments / total number of samples \qquad ( Eq . 1 )$$

$$SPEC = T N / ( T N + F P ) ; is a true negative rate \qquad ( Eq . 2 )$$

$$SEN = T P / ( T P + F N ) ; is true positive rate \qquad ( Eq . 3 )$$

### Discussion

Cells have chemical features that set them apart, but those features can be subtle and difficult to detect. However, these subtle chemical differences are identified by FTIR spectral phenotyping. This example shows that the spectral imaging approach reproducibly and reliably predicts control or disease classification using an FTIR signature as a biomarker. Not only did it accurately predict disease class, but the FTIR signatures were able to discriminate between control and disease astrocytes from animals as early as 2 days after birth. At this stage, WT and HD animals had distinct genotypes, but the number of neurons, morphology and antibody staining patterns in the brain were equivalent. In the absence of obvious pathology, FTIR signatures correctly classed them as control or disease. Spectral phenotyping can provide a mechanism to detect and track even subtle changes in a cell's chemical states with high probability at early stages of disease progression. Classification by FTIR is possible using standard FTIR equipment which is available for use in universities and in hospital environments. The FTIR signature is robust and applies across disease types, cell types, and species in these proof of principle experiments. Spectral phenotyping can be used to broadly identify cellular changes of state such as those that occur in disease, viral infection, drug exposure, and embryonic development.

More than a decade of ground-breaking work has catapulted FTIR imaging as a powerful new tool with great promise for clinical applications. Recent technological advances have and will continue to improve the technique. For example, synchrotron radiation is 100 to 1000 times brighter than conventional thermal IR light, providing a better spatial resolution and leading to unprecedented chemical probing of live cells. The use of Quantum Cascade Lasers (QCLs) offers the ability to scan specific wavenumbers of interest, which decrease the time of acquisition. Submicrometer spatial resolution is now achievable by mid-IR photothermal microspectroscopy with commercially available bench top instruments. This method has been used to observe amyloid protein aggregates at subcellular level, along the neurites and dendritic spines of neurons. Thus, FTIR spectroscopy is increasing in its capabilities to classify both fixed and live cells.

With respect to clinical application, the spectral phenotyping method offers three advances. First, this example shows that spectral phenotyping can accurately classify disease states before manifest symptoms. If disease pathology is well understood, FTIR spectroscopy is not needed to classify post-mortem tissue at the end of life. As an early biomarker, however, spectral phenotyping would be invaluable in disease predictions for asymptomatic patients during life or for the many diseases where a diagnosis is difficult or unclear. As an example, in the absence of cognitive decline, a diagnosis of a pre-symptomatic AD patient is tentative and disease candidates are determined based on low levels of amyloid-beta peptide in the blood or in MRI brain images. Yet, a diagnosis is uncertain since these aggregates are also present in the normal aging population. Similarly, infrared spectroscopy has been useful in examining the conformation and structure of mature aggregates, but they are not present in HD fibroblasts or in HD mouse models at early stages of disease. Second, using segmentation and UMAP analysis, robust disease predictions were achievable. UMAP, unlike PCA, is a non-linear dimension reduction method. UMAP prioritizes distances, i.e., the closeness of neighbors, and maximizes the separation among samples, allowing robust clustering for a larger number of samples. Although whole cells or nuclei have been common regions for feature extraction by scientists, this example shows that subcellular segmentation can be important for the analysis algorithm since misclassification can occur if the correct segments are not used. Third, each signature comprises hundreds of cells allowing a robust signature and the analysis is relatively rapid and economical. With data in hand for segmentation, the processing time of 16384 spectra contained in one FOV on a local computer was around 160 ms. The entire acquisition time for hundreds of cells, required for robust classification, is most often complete in under an hour with an FPA detector, and off-line analysis is complete in two hours. High throughput is possible using an assembly line approach. Moreover, the speed of FTIR imaging will improve further with technological advances, and that the use of IR spectral signatures will increase throughput and will outpace other approaches as a basis for accurate disease classification.

The importance of a biomarker for disease predictions cannot be overstated. There is a desperate need to develop therapeutic compounds, but there is no classification criteria by which to judge when to start or stop treatment, which is also costly and time consuming. Thus, the gap between the incidence of disease and the ability to treat patients is growing exponentially. Although the use of serum samples in FTIR analysis has advanced considerably, this example shows that surrogate skin cells at early ages can be used for reliable disease predictions of neurological and neurodegenerative diseases. Although they have distinct functions from that of neurons, peripheral cells such as fibroblasts are stable, maintain the genetic background of the patient, and the chemical alterations which track with disease are detectable by FTIR spectroscopy. The wide availability of lymphoblasts, fibroblasts, and induced pluripotent stem cells (iPSCs) provide new opportunities to collect samples from living patients with neurological disorders, and track disease endpoints at very early biological states with minimum discomfort. A biomarker which is sensitive to disease progression and its reversal would be valuable in that early detection would lower the cost and time of treatment by predicting a treatment window.

Spectral phenotyping described in this example has highly accuracy in the age and gender matched samples and controls used in this example. These results suggest that spectral phenotyping holds promise as a clinically relevant biological tool. Factors such as lifestyle, ethnicity and medical background may introduce more variability. More extensive analysis using additional statistical or clinical parameters can be performed to retain a robust disease prediction by FTIR spectroscopy. Nonetheless, classification using FTIR signatures is accurate, and the measurements require minimal sample preparation and no a priori knowledge of the sample, which can be highly useful for unbiased disease classification (e.g., disease versus non-disease). Signature specificity can be an important consideration. In theory, millions of combinatorial signatures are possible in the mid-IR range between 4000 and 800 cm⁻¹. However, there will be overlap and redundancy among spectral features, possibly placing limits to the number of discrete signatures. For example, lipids can change in many disorders or abnormalities, and therefore are not specific to a particular disease. There may be limits to signature “uniqueness”. The patients analyzed in this example from the Coriell repository are unrelated and therefore, are unlikely to have shared the same lifestyle, have the same cholesterol levels, or the same diet, but disease predictions for both HD and AD populations are robust when compared to controls. Although both diseases are associated with lipid abnormalities and both have lipid features that contribute significantly to their signature spectra, when compared to each other, AD and HD from distinct groups that do not overlap (FIG. 12C).

In summary, spectral phenotyping by FTIR spectroscopy meets the ever-increasing demand to measure unperturbed, native states, with wide ranging applications in cell biology, diagnoses, and predictive biology. The approach enables prediction of cells that are diseased or behave differently with age, type or during disease progression, all of which have been difficult to achieve reliably using other methods.

### Example 2

An infrared spectral biomarker discriminates among neurological diseases and diseases that are not neurodegenerative

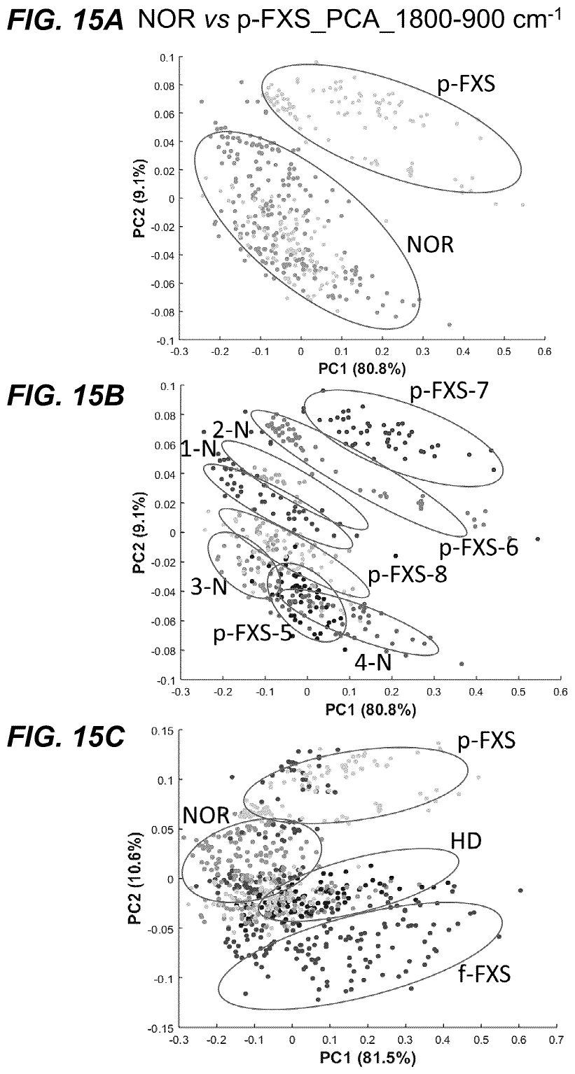

FIGS. 15A-15C. FTIR discriminates among neurological disease. FIGS. 15A-15B. Representative PCA analysis of the FTIR signature spectra of human fragile X premutation (P, yellow in FIG. 15A) and control fibroblasts (green in FIG. 15A), as labeled. FIG. 15C. Combined plot of Fragile X premutation syndrome of premutation (P, yellow) and full mutation (F, red), compared to normal (NOR green) fibroblasts and to unrelated HD fibroblasts (blue), as disease groups (color coded). Fragile X is a systemic disease with neurological disease symptoms. It is generated from an expansion of repeating CGG in the intron of the FMR-1 gene: normal level is below 50 CGG repeats; premutation carriers (55-200) are susceptible to disease; full mutation is disease range is >200 repeats and expresses full disease phenotype.

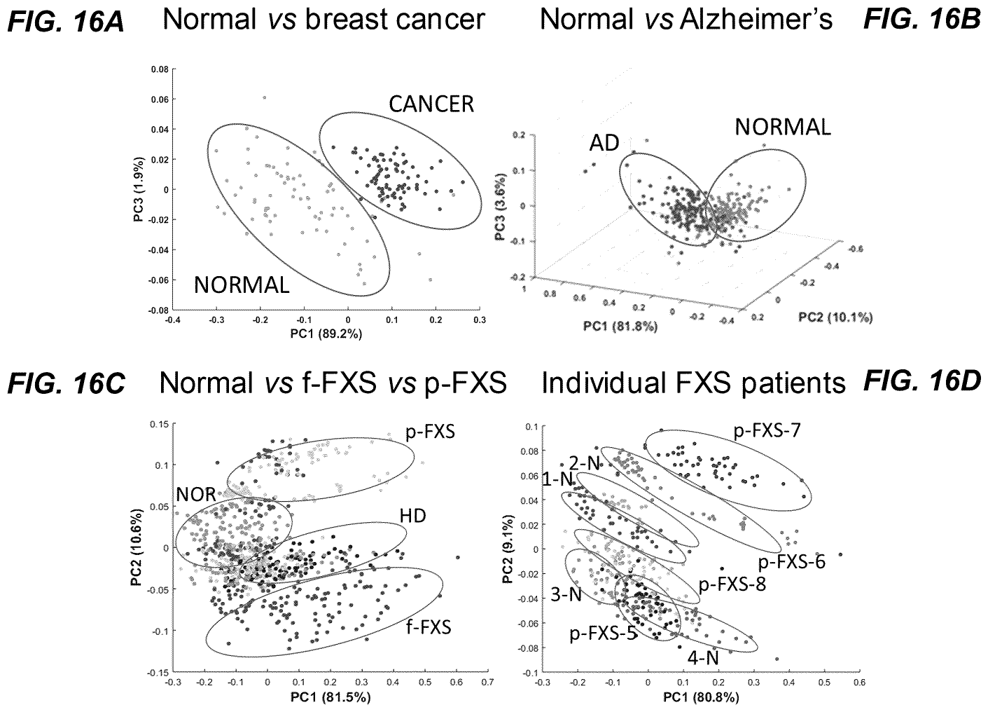

FIGS. 16A-16D. FTIR discriminates among other disease that are not neurodegenerative. Representative PCA analysis of the FTIR signature spectra of (FIG. 16A) human normal epithelial cells and breast cancer epithelial cells; and (FIG. 16B) human Alzheimer's fibroblasts. Red is disease and green are control. FIG. 16C. Combined plot of Fragile X premutation syndrome of (P, premutation yellow), and (F, full mutation), compared to normal (NOR green) fibroblasts and to unrelated HD fibroblasts (blue), as disease groups (color coded). Fragile X is a systemic disease with neurological disease symptoms. It is generated from an expansion of repeating CGG in the intron of the FMR-1 gene: normal level is below 50 CGG repeats; premutation carriers (55-200) are susceptible to disease; full mutation is disease range is >200 repeats and expresses full disease phenotype. FIG. 16D. PCA of Fragile X patients and controls plotted as individuals. Each individual patient and control is color coded. Spectral phenotyping has applications for personalized medicine, although more detailed analysis will be needed to sort them discretely.

### Execution Environment

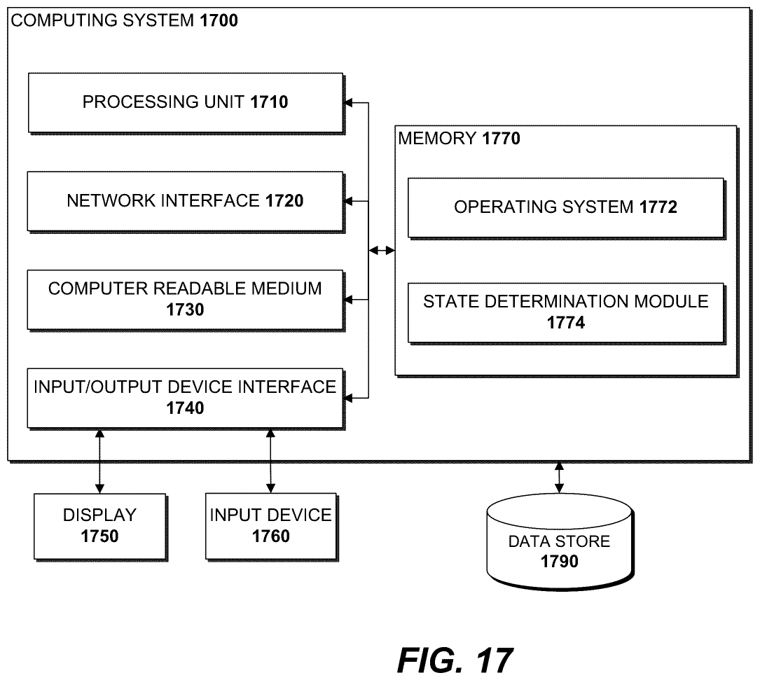

FIG. 17 depicts a general architecture of an example computing device 1700 that can be used in some embodiments to execute the processes and implement the features described herein. The general architecture of the computing device 1700 depicted in FIG. 17 includes an arrangement of computer hardware and software components. The computing device 1700 may include many more (or fewer) elements than those shown in FIG. 17. It is not necessary, however, that all of these generally conventional elements be shown in order to provide an enabling disclosure. As illustrated, the computing device 1700 includes a processing unit 1710, a network interface 1720, a computer readable medium drive 1730, an input/output device interface 1740, a display 1750, and an input device 1760, all of which may communicate with one another by way of a communication bus. The network interface 1720 may provide connectivity to one or more networks or computing systems. The processing unit 1710 may thus receive information and instructions from other computing systems or services via a network. The processing unit 1710 may also communicate to and from memory 1770 and further provide output information for an optional display 1750 via the input/output device interface 1740. The input/output device interface 1740 may also accept input from the optional input device 1760, such as a keyboard, mouse, digital pen, microphone, touch screen, gesture recognition system, voice recognition system, gamepad, accelerometer, gyroscope, or other input device.

The memory 1770 may contain computer program instructions (grouped as modules or components in some embodiments) that the processing unit 1710 executes in order to implement one or more embodiments. The memory 1770 generally includes RAM, ROM and/or other persistent, auxiliary or non-transitory computer-readable media. The memory 1770 may store an operating system 1772 that provides computer program instructions for use by the processing unit 1710 in the general administration and operation of the computing device 1700. The memory 1770 may further include computer program instructions and other information for implementing aspects of the present disclosure.

For example, in one embodiment, the memory 1770 includes a state determination module 1774 for determining the state (e.g., phenotype, disease state, treatment responsiveness) of a subject using the spectral genotyping method of the present disclosure. In addition, memory 1770 may include or communicate with the data store 1790 and/or one or more other data stores that store input, intermediate results, and/or output of the spectral genotyping method described herein, such as FTIR spectra (e.g., quality-tested spectra, pre-processed spectra) and the state determined for the subject.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A method for determining a state of a test subject, comprising:
generating a plurality of reference Fourier transform infrared spectroscopy (FTIR) spectra for each of a plurality of reference samples, wherein the plurality of reference samples comprises a plurality of first reference samples obtained from first reference subjects known to be in a first state and a plurality of second reference samples obtained from reference subjects known to be a second state;
determining an average reference FTIR spectrum of the plurality of reference FTIR spectra for each of the plurality of reference samples;
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples and the average test FTIR spectrum into a first cluster and a second cluster corresponding to the first state and the second state, respectively; and
determining the test subject is in the first state or the second state based on whether the average test FTIR spectrum is in the first cluster or the second cluster.

2. A method for determining a state of a test subject, comprising:
generating a plurality of reference Fourier transform infrared spectroscopy (FTIR) spectra for each of a plurality of reference samples, wherein the plurality of reference samples comprises a plurality of first reference samples obtained from first reference subjects known to be in a first state and a plurality of second reference samples obtained from reference subjects known to be a second state;
determining an average reference FTIR spectrum of the plurality of reference FTIR spectra for each of the plurality of reference samples;
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples into a first cluster and a second cluster corresponding to the first state and the second state, respectively, in a reduced dimensionality space; and
determining the test sample is in the first state or the second state based on a first distance between the average test FTIR spectrum and the first cluster and a second distance between the average test FTIR spectrum and the second cluster in the reduced dimensionality space.

3. The method of any one of claims 1-2, wherein each of the plurality of reference samples and the test sample comprises about 100 cells to about 1000 cells, and/or wherein each of the plurality of reference samples and the test sample comprises about the same number of cells.

4. The method of any one of claims 1-3, wherein the sample comprises a tissue sample, optionally wherein the tissue sample is about 10 m in thickness, optionally wherein the tissue sample comprises one layer of cells.

5. The method of any one of claims 1-4, wherein the sample comprises surrogate cells, optionally wherein the surrogate cells comprise accessible cell types, epithelial cells, fibroblasts, lymphoblasts, peripheral cells, non-neural cells, buccal cells, induced pluripotent stem cells, or a combination thereof.

6. The method of any one of claims 1-5, wherein the plurality of first reference samples comprises at least 10 samples, and/or wherein the plurality of second reference samples comprises at least 10 samples.

7. The method of any one of claims 1-6, wherein the plurality of reference samples and the test sample comprise fixed cells on slides.

8. The method of any one of claims 1-7, wherein the plurality of reference samples and the test sample were prepared in an identical manner, and/or wherein the plurality of reference FTIR spectra for each of the plurality of samples and the plurality of test FTIR spectra were captured in an identical manner.

9. The method of any one of claims 1-8, wherein the slides comprise Calcium fluoride (CaF₂) or silicon (Si) slides, wherein the slides comprise no coating, wherein the slides comprises a coating, wherein the coating comprises poly-L-ornithine (PLO), and/or wherein the coating comprises wet PLO or dry PLO.

10. The method of any one of claims 1-9, wherein the slides were previously stored at room temperature or −80° C. for up to two weeks.

11. The method of any one of claims 1-10, wherein generating the plurality of reference FTIR spectra for each of the plurality of samples and the plurality of test FTIR spectra comprises capturing the plurality of reference FTIR spectra for each of the plurality of samples and the plurality of test FTIR spectra at room temperature or −80° C.

12. The method of any one of claims 1-11,
wherein the first state comprises a first phenotype, and wherein the second state comprises a second phenotype,
wherein the first state is non-responsiveness to a treatment of a disease, and wherein the second state is responsiveness to the treatment of the disease,
wherein the first state is a non-diseased state, and wherein the second state is a diseased state, and/or
wherein the disease is a disease subtype, optionally wherein the disease is a neurological disease, a neurodegenerative disease, a late onset disease, or a cancer, optionally wherein the neurological disease or the neurodegenerative disease comprises Alzheimer's disease, Huntington's disease, or Fragile X syndrome.

13. The method of any one of claims 1-12, wherein the one or more characteristics of the test subject and the reference subjects that are matched comprise age, gender, lifestyle, diet, health, ethnicity, and/or medical background.

14. The method of any one of claims 1-13, wherein the second reference subjects have no symptoms or have no overt symptoms.

15. The method of any one of claims 1-14, wherein the plurality of reference FTIR spectra, the average reference FTIR spectra, the plurality of test FTIR spectra, and the average test FTIR spectra comprise second derivative absorbance spectra.

16. The method of any one of claims 1-15, wherein the plurality of reference FTIR spectra, the average reference FTIR spectra, the plurality of test FTIR spectra, and the average test FTIR spectra comprise spectra between 3050-2800 cm⁻¹ and/or 1800-900 cm⁻¹.

17. The method of any one of claims 1-16, wherein the plurality of reference FTIR spectra for each of the plurality of reference samples and the plurality of test FTIR spectra comprise FTIR spectra generated from whole cells.

18. The method of any one of claims 1-17, wherein the plurality of reference FTIR spectra for each of the plurality of reference samples and the plurality of test FTIR spectra comprise FTIR spectra generated from cytoplasm of cells.

19. The method of claim 18, comprising segmenting the plurality of reference FTIR spectra for each of the plurality of reference samples and the plurality of test FTIR spectra to determine reference FTIR spectra of the plurality of reference FTIR spectra for each of the plurality of reference samples and test FTIR spectra of the plurality FTIR spectra generated from cytoplasm of cells, wherein the segmenting is based on integrated absorbance frequencies between 1670-1630 cm⁻¹.

20. The method of any one of claims 1-19, comprising quality testing the plurality of reference FTIR spectra for each of the plurality of samples and the plurality of test FTIR spectra to generate a plurality of quality-tested, reference FTIR spectra for each of the plurality of samples and the plurality of quality-tested, test FTIR spectra, wherein determining the average reference FTIR spectrum of the plurality of reference FTIR spectra for each of the plurality of reference samples comprises determining an average reference FTIR spectrum of the plurality of quality-tested, reference FTIR spectra for each of the plurality of reference samples, and wherein determining the average test FTIR spectrum comprises determining the average test FTIR spectrum of the plurality of quality-tested, test FTIR spectra.

21. The method of any one of claims 1-20, comprising pre-processing the plurality of reference FTIR spectra for each of the plurality of samples and the plurality of test FTIR spectra to generate a plurality of pre-processed, reference FTIR spectra for each of the plurality of samples and the plurality of pre-processed, test FTIR spectra, wherein determining the average reference FTIR spectrum of the plurality of reference FTIR spectra for each of the plurality of reference samples comprises determining an average reference FTIR spectrum of the plurality of pre-processed, reference FTIR spectra for each of the plurality of reference samples, and wherein determining the average test FTIR spectrum comprises determining the average test FTIR spectrum of the plurality of pre-processed, test FTIR spectra, optionally wherein pre-processing comprises smoothing, baseline correction, spectral contrast optimization, and/or vector normalization.

22. The method of any one of claims 1-21, wherein the plurality of reference FTIR spectra for each of the plurality of reference samples and the plurality of test FTIR spectra comprise normalized second derivative spectra.

23. The method of any one of claims 1-22,
wherein clustering the average reference FTIR spectra of the plurality of reference samples comprises dimensionality reduction,
wherein clustering the average reference FTIR spectra of the plurality of reference samples comprises unsupervised clustering, and/or
wherein the unsupervised clustering comprises Principal Component Analysis (PCA) and Uniform Manifold Approximation and Projection (UMAP) analysis.

24. The method of any one of claims 1-23, wherein a Silhouette score of the test sample being determined to be in the first state or the second state is about 0.4 to 0.9, wherein sensitivity of the test sample being determined to be in the first state or the second state is at least 0.8, wherein specificity of the test sample being determined to be in the first state or the second state is at least 0.8, and/or wherein accuracy of the test sample being determined to be in the first state or the second state is at least 0.8.

25. The method of any one of claims 1-24, wherein the average test FTIR spectrum is in the first cluster if a first distance between the average test FTIR spectrum and the first cluster is shorter than a second distance between the average test FTIR spectrum and the second cluster, and wherein the average test FTIR spectrum is in the first cluster if a first distance between the average test FTIR spectrum and the first cluster is longer than a second distance between the average test FTIR spectrum and the second cluster.

26. The method of any one of claims 1-25, wherein the first distance between the average test FTIR spectrum and the first cluster comprises the first distance between the average test FTIR spectrum and a center of the first cluster, and wherein the second distance between the average test FTIR spectrum and the second cluster comprises the second distance between the average test FTIR spectrum and a center of the second cluster.

27. The method of any one of claims 1-25, wherein the first distance between the average test FTIR spectrum and the first cluster comprises the first distance between the average test FTIR spectrum and k-nearest neighbors of the first cluster, and wherein the second distance between the average test FTIR spectrum and the second cluster comprises the second distance between the average test FTIR spectrum and k-nearest neighbor of the second cluster, optionally wherein k is 10.

28. A system for determining a state of a test subject comprising:
non-transitory memory configured to store executable instructions; and
a hardware processor in communication with the non-transitory memory, the hardware processor programmed by the executable instructions to perform:
generating a plurality of reference Fourier transform infrared spectroscopy (FTIR) spectra for each of a plurality of reference samples, wherein the plurality of reference samples comprises a plurality of first reference samples obtained from first reference subjects known to be in a first state and a plurality of second reference samples obtained from reference subjects known to be a second state;
determining an average reference FTIR spectrum of the plurality of reference FTIR spectra for each of the plurality of reference samples;
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples and the average test FTIR spectrum into a first cluster and a second cluster corresponding to the first state and the second state, respectively; and
determining the test subject is in the first state or the second state based on whether the average test FTIR spectrum is in the first cluster or the second cluster.

29. A system for determining a state of a test subject comprising:
non-transitory memory configured to store executable instructions and an average reference Fourier transform infrared spectroscopy (FTIR) spectrum of a plurality of reference FTIR spectra for each of a plurality of reference samples, wherein the plurality of reference samples comprises a plurality of first reference samples obtained from first reference subjects known to be in a first state and a plurality of second reference samples obtained from reference subjects known to be a second state; and
a hardware processor in communication with the non-transitory memory, the hardware processor programmed by the executable instructions to perform:
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples and the average test FTIR spectrum into a first cluster and a second cluster corresponding to the first state and the second state, respectively; and
determining the test subject is in the first state or the second state based on whether the average test FTIR spectrum is in the first cluster or the second cluster.

30. A system for determining a state of a test subject comprising:
non-transitory memory configured to store executable instructions; and
a hardware processor in communication with the non-transitory memory, the hardware processor programmed by the executable instructions to perform:
generating a plurality of reference Fourier transform infrared spectroscopy (FTIR) spectra for each of a plurality of reference samples, wherein the plurality of reference samples comprises a plurality of first reference samples obtained from first reference subjects known to be in a first state and a plurality of second reference samples obtained from reference subjects known to be a second state;
determining an average reference FTIR spectrum of the plurality of reference FTIR spectra for each of the plurality of reference samples;
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples into a first cluster and a second cluster corresponding to the first state and the second state, respectively, in a reduced dimensionality space; and
determining the test subject is in the first state or the second state based on a first distance between the average test FTIR spectrum and the first cluster and a second distance between the average test FTIR spectrum and the second cluster.

31. A system for determining a state of a test subject comprising:
non-transitory memory configured to store executable instructions and an average reference Fourier transform infrared spectroscopy (FTIR) spectrum of a plurality of reference FTIR spectra for each of a plurality of reference samples, wherein the plurality of reference samples comprises a plurality of first reference samples obtained from first reference subjects known to be in a first state and a plurality of second reference samples obtained from reference subjects known to be a second state; and
a hardware processor in communication with the non-transitory memory, the hardware processor programmed by the executable instructions to perform:
generating a plurality of test FTIR spectra for a test sample obtained from a test subject, wherein one or more characteristics of the test subject and the reference subjects are matched;
determining an average test FTIR spectrum of the plurality of test FTIR spectra for the test sample;
clustering the average reference FTIR spectra of the plurality of reference samples into a first cluster and a second cluster corresponding to the first state and the second state, respectively, in a reduced dimensionality space; and
determining the test subject is in the first state or the second state based on a first distance between the average test FTIR spectrum and the first cluster and a second distance between the average test FTIR spectrum and the second cluster.
